(19) United States
(12) Patent Application Publication
(10) Pub. No.: US 2026/0324983 A1
(43) Pub. Date: Oct. 22, 2026

(54) CORE BODY TEMPERATURE

(71) Applicant: Whoop, Inc., Boston, MA (US)

(72) Inventors: Arvind Krishnamachari Venkatesh, Lexington, MA (US); Alejandra Moreno, Brookline, MA (US)

(21) Appl. No.: 19/655,308

(22) Filed: Apr. 22, 2026

Related U.S. Application Data
(60) Provisional application No. 63/792,512, filed on Apr. 22, 2025.

(51) Int. Cl. A61B 5/01; A61B 5/00

(52) U.S. Cl. CPC A61B 5/01; A61B 5/681; A61B 2560/0214; A61B 2560/0223

(57) ABSTRACT

An arrangement of temperature sensors within a wearable device can be used in combination with a controllable heat source to evaluate heat flux through the device and develop a lumped thermal resistance model for estimating a core body temperature for the wearer. As a significant advantage, a charging circuit within the wearable device, or removably attachable to the device, e.g., for a wireless charging battery, can be used as the controllable heat source to characterize parameters of a thermal model for the wearable device in a manner that supports calculations of the core body temperature.

### RELATED APPLICATIONS

This application claims priority to U.S. Prov. App. No. 63/792,512, filed on Apr. 22, 2025, the entire content of which is hereby incorporated by reference.

### BACKGROUND OF THE INVENTION

Non-invasive and accurate measurements of core body temperature present a significant technical challenge. Core body temperature is different from the temperature measured or sensed at skin depth, and is often measured through invasive procedures using, e.g., pulmonary catheters or rectal sensors. Although effective, these methods are inconvenient, uncomfortable, and difficult to deploy in wearable devices for continuous monitoring.

Non-invasive technologies for core temperature measurement have been disclosed, such as the TCore device from the Drägerwerk AG & Co. of Lübeck, Germany. The TCore device employs a self-adhesive sensor that is placed on the patient's forehead. Although these systems can work well, they are cumbersome and typically require some level of clinical training for correct use. Moreover, the TCore system is adhesively attached to one's forehead, which may render the system difficult or inconvenient for continuous use. Other devices for non-invasive measures of core body temperature have been disclosed in Huang et al.; A Wearable Thermometry for Core Body Temperature Measurement and Its Experimental Verification; IEEE Journal of Biomedical and Health Informatics, Vol. 21, No. 3, (May 2017). These devices also rely on a heat flow from the skin and are worn on the forehead.

There remains a need for core body monitoring techniques suitable for wearable physiological monitors, such as wrist-worn fitness monitors used by athletes and others to track physiological metrics.

### SUMMARY OF THE INVENTION

An arrangement of temperature sensors within a wearable device can be used in combination with a controllable heat source to evaluate heat flux through the device and develop a lumped thermal resistance model for estimating a core body temperature for the wearer. As a significant advantage, a charging circuit within the wearable device, or removably attachable to the device, e.g., for a wireless charging battery, can be used as the controllable heat source to characterize parameters of a thermal model for the wearable device in a manner that supports calculations of the core body temperature.

In one aspect, there is disclosed herein a system including: a wearable monitor including a housing configured to be worn against a skin of a user, the housing including a first temperature sensor positioned to obtain a temperature at the skin; a power supply removably and replaceably coupled to the housing of the wearable monitor, the power supply including a second temperature sensor, the power supply operable in a first mode as a recharging battery to provide electrical power to recharge the wearable monitor, and the power supply operable in a second mode to generate controlled heat for injection through a thermal stack extending from the power supply to the skin of the user; and processing circuitry configured to: operate the power supply in the second mode to provide a controlled thermal excitation, receive temperature information from the first temperature sensor and the second temperature sensor indicative of a temperature response of the thermal stack to the controlled thermal excitation, determine a heat flux through the thermal stack based on the temperature response, and estimate an internal body temperature of the user based on the heat flux through the thermal stack.

The processing circuitry may be configured to estimate the internal body temperature using a lumped thermal model calibrated based on a number of measurements of thermal flux through the housing under a number of operating conditions. The lumped thermal model may represent the user, the housing, and the power supply as elements of the thermal stack having corresponding thermal resistances. The system may include a temperature sensor positioned to measure an ambient temperature, wherein the lumped thermal model is calibrated based at least in part on a second heat flux from the housing to an ambient environment. The processing circuitry may be configured to estimate the internal body temperature based on a time constant derived from the temperature response. The processing circuitry may initiate one or more heat injections from the power supply and calculates the time constant based on a rise time for a temperature at one or more of the first temperature sensor and the second temperature sensor. The processing circuitry may be configured to detect a steady state of the temperature response to the controlled thermal excitation and to estimate the internal body temperature based on one or more temperature measurements at the steady state. The power supply may include a second sensor for measuring a temperature and the second sensor is disposed on a second side of the processing circuitry opposite a first side of the processing circuitry facing the skin of the user. The processing circuitry may store one or more parameters representative of nodes of a lumped thermal model for estimating the internal body temperature, the lumped thermal model including at least a first thermal conductivity associated with the power supply and a second thermal conductivity associated with the housing. The system may include a third temperature sensor on a second side of the housing opposing the first temperature sensor and a fourth temperature sensor on a second side of the power supply opposing the second temperature sensor. The housing may include a heat flux transducer for measuring a housing heat flux therethrough.

In another aspect, there is disclosed herein a computer program product for estimating an internal body temperature of a user with a wearable monitor, the computer program product comprising computer-executable code embodied in a non-transitory computer-readable medium that, when executing on one or more computing devices, causes the one or more computing devices to perform the steps of: storing parameters for a lumped thermal model of the wearable monitor; controlling a recharging battery removably coupled to the wearable monitor to provide a controlled thermal excitation to the wearable monitor; while providing the controlled thermal excitation to the wearable monitor, measuring a skin temperature of the user where the wearable monitor contacts a skin of the user; while providing the controlled thermal excitation to the wearable monitor, measuring a heat flux through the wearable monitor between a surface of the wearable monitor contacting the skin and a location between the surface of the wearable monitor and the recharging battery; and estimating the internal body temperature of the user by applying the skin temperature and the heat flux to the lumped thermal model.

The parameters for the lumped thermal model may include a thermal conductivity of at least one of a body of the user, the wearable monitor, and the recharging battery. The code may include code that causes the one or more computing devices to perform the step of calibrating the lumped thermal model based on one or more temperature measurements under controlled conditions including predetermined configurations of the wearable monitor and the recharging battery. The one or more temperature measurements may include one or more temperature measurements while the recharging battery is coupled to the wearable monitor and the wearable monitor is not worn by the user. The one or more temperature measurements may include one or more temperature measurements while the wearable monitor is placed for use on the skin without the recharging battery. The wearable monitor may be wirelessly coupled to the recharging battery for power and communications. The code may include code that causes the one or more computing devices to perform the step of initiating the controlled thermal excitation from the wearable monitor through a wireless communications interface between the wearable monitor and the recharging battery.

In another aspect, there is disclosed herein a method for estimating an internal body temperature of a user, the method comprising: positioning a first side of a wearable monitor against a skin of the user; removably coupling a power supply to a second side of the wearable monitor opposing the first side; while providing a controlled thermal excitation to the wearable monitor with the power supply coupled to the wearable monitor, performing the steps of: measuring a skin temperature of the user where the wearable monitor contacts the skin, and measuring a heat flux through the wearable monitor; and estimating the internal body temperature of the user based on the skin temperature and the heat flux. Measuring the heat flux may include measuring the heat flux with a plurality of temperature sensors within the wearable monitor.

### BRIEF DESCRIPTION OF DRAWINGS

The systems and methods described herein are set forth in the appended claims. However, for the purpose of explanation of these systems and methods, several embodiments are set forth in the following figures and the related description.

FIG. 1 shows a physiological monitoring device.

FIG. 2 shows a physiological monitoring system.

FIG. 3 shows a sensing system.

FIG. 4A shows examples of physiological monitoring devices.

FIG. 4B shows examples of physiological monitoring devices.

FIG. 4C shows examples of physiological monitoring devices.

FIG. 5 shows a smart garment system.

FIG. 6 depicts a functional block diagram of a physiological monitoring device with core temperature sensing.

FIG. 7 depicts a cross-section of a physiological monitoring device with core temperature sensing.

FIG. 8 depicts a flow of heat through a system including a wearable device and a removable power supply.

FIG. 9 depicts a heat flow model for a wearable device positioned for monitoring on a user.

FIG. 10 shows a method for estimating core temperature with a wearable device.

### DETAILED DESCRIPTION

In the following description, numerous details are set forth for the purpose of explanation. In particular, improved systems and methods for measuring core body temperature are disclosed. However, one of ordinary skill in the art will realize that the embodiments described herein may be practiced without the use of these specific details. Further, for clarity, well-known structures and devices are shown in block diagram form to not obscure the description with unnecessary detail.

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “user” as used herein, refers to any type of animal, human or non-human, whose physiological information may be monitored using an exemplary wearable physiological monitoring device and/or system.

The term “continuous,” as used herein in connection with heart rate data, refers to the acquisition of heart rate data at a sufficient frequency to enable detection of individual heartbeats, and also refers to the collection of heart rate data over extended periods such as an hour, a day or more (including acquisition throughout the day and night), etc. More generally, with respect to physiological signals that might be monitored by a wearable device, “continuous” or “continuously” will be understood to mean continuously at a rate and duration suitable for the intended time-based processing, and physically at an inter-periodic rate (e.g., multiple times per heartbeat, respiration, and so forth) sufficient for resolving the desired physiological characteristics such as heart rate, heart rate variability, heart rate peak detection, pulse shape, and so forth. Continuous monitoring should also be understood to include periodic sampling at any suitable interval, duration, and frequency. Thus, for example, continuous monitoring may include measuring a user's body temperature once every ten minutes, or monitoring heart activity by alternately sampling the heart rate for a minute and then pausing sampling for a minute, e.g., to conserve power or memory at times when the measured heart rate indicates that the user is at rest. Sampling may also be dynamic based on sensor input, for example, increasing the sampling rate when signal variability increases, or during periods of relatively higher motion, or based on user input.

At the same time, continuous monitoring is not intended to exclude ordinary data acquisition interruptions, such as temporary displacement of monitoring hardware due to sudden movements, changes in external lighting, loss of electrical power, physical manipulation and/or adjustment by a wearer, physical displacement of monitoring hardware due to external forces, and so forth. It will also be noted that heart rate data or a monitored heart rate, in this context, may more generally refer to raw sensor data, such as optical intensity signals, or processed data therefrom, such as heart rate data, signal peak data, heart rate variability data, or any other physiological or digital signal suitable for recovering heart rate information as contemplated herein. Furthermore, such heart rate data may generally be captured over some historical period that can be subsequently correlated to various other data or metrics related to, e.g., sleep states, recognized exercise activities, resting heart rate, maximum heart rate, and so forth.

The term “computer-readable medium,” as used herein, refers to any non-transitory storage media, such as storage hardware, storage devices, computer memory that may be accessed by a controller, a microcontroller, a microprocessor, a computational system, or the like, or any other module or component of a computational system to encode thereon computer-executable instructions, software programs, and/or other data. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), virtual or physical computer system memory, physical memory hardware, such as random access memory (such as DRAM, SRAM, EDO RAM), and so forth. Although not depicted, any of the devices or components described herein may include a computer-readable medium or other memory for storing program instructions, data, and the like.

As used herein, the phrase core body temperature (CBT) is intended to refer to the thermal operating temperature of the internal structures of a subject, such as a subject's deep tissues and organs, including the brain, the heart, and the abdominal viscera. More generally, the CBT or deep body temperature will be understood as the relatively homogeneous internal temperature of the human body. Unlike peripheral or shell temperature (e.g., skin), CBT is the physiologically regulated temperature maintained by the hypothalamus to ensure homeostatic metabolic function. The physical quantity measured is the internal thermal energy or heat content of the blood and deep-seated tissues in a state of thermal equilibrium. In a clinical or physiological context, this represents the temperature of the blood circulating through the pulmonary artery, which provides the most accurate reflection of the body's integrated thermal state. Standard baseline CBT for a healthy human adult is typically defined within the range of 36.5-37.5 °C (97.7-99.5 °F), though this is subject to circadian rhythms and individual physiological variance. Other phrases, such as “internal temperature”, “internal body temperature”, “core temperature,” and the like, are also variously used herein, and are intended to have similar scope unless explicitly stated otherwise.

Current techniques for determining CBT are categorized into invasive (direct) and non-invasive (proxy) methods. For example, the clinical “gold standard” is an invasive technique using a Pulmonary Artery (PA) Catheter. This provides a direct thermistor-based measurement of the blood temperature within the heart's outflow tract. Other high-accuracy invasive sites include the distal esophagus (reflecting retrocardiac temperature) and the urinary bladder (when urine flow is adequate). Rectal thermometry is also a widely accepted clinical standard for core temperature, though it is subject to significant temporal lag during rapid thermal transitions (e.g., during exercise or cooling). Common practical techniques include tympanic membrane (infrared sensing of the ear canal), oral (sublingual), axillary (underarm), and temporal artery (forehead) measurements. While non-invasive, these methods estimate core temperature based on accessible peripheral sites.

There exists significant variability in current measurement techniques. For example, a natural thermal gradient exists between the body core and the skin surface. Peripheral sites (oral, axillary) are often 0.3-0.9 °C lower than internally measured CBT and are highly susceptible to ambient environmental conditions. Furthermore, certain sites, particularly rectal measurements, exhibit a “thermal inertia” where changes in the true core temperature are not reflected in the measurement for several minutes, leading to inaccuracies during acute medical events or high-intensity activity. Also, non-invasive methods, such as tympanic or temporal scanning, are highly dependent on sensor placement, probe-to-tissue contact, and the presence of moisture (sweat) or cerumen (earwax), often resulting in low inter-device reliability. As such, there remains a need for wearable temperature-monitoring techniques that can estimate deep-body temperature in a practical form factor suitable for extended physiological monitoring.

To address these challenges, multiple temperature measurements can be taken at multiple points in a lumped thermal model of heat flux passing from a physical body through one or more components of a wearable monitoring system, which permits modeling of heat flux, rather than just surface temperature, in order to support more accurate inferences about core body temperature. In some embodiments, a wearable device uses a controlled thermal excitation and a heat-flux-based estimation process. A temperature sensor at a skin-facing portion of the wearable device can obtain a skin-related temperature, while a thermally coupled power supply or other controllable heat source can be driven to a selected temperature or with a predetermined temperature profile and monitored with one or more additional temperature sensors. Based on the resulting temperature response through the thermal stack of the wearable device, processing circuitry can estimate heat flux through the device and apply a thermal model, optionally using calibration data and ambient-condition measurements, to determine an estimate of the wearer's deep-body temperature. In this manner, the wearable device can support non-invasive deep-body temperature monitoring using a compact architecture that remains compatible with broader physiological monitoring functions.

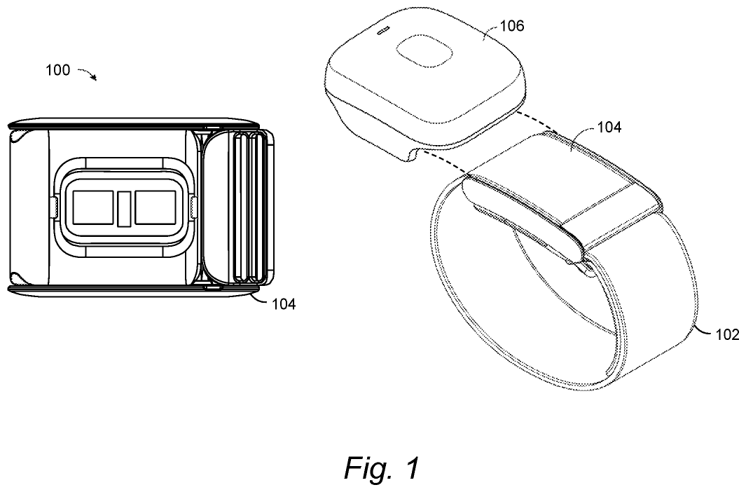

FIG. 1 shows a physiological monitoring system. The system 100 may include a wearable monitor 104 that is configured for physiological monitoring. The system 100 may also include a removable and replaceable battery 106 for recharging the wearable monitor 104. The wearable monitor 104 may include a strap 102 or other retaining system(s) for securing the wearable monitor 104 in a position on a wearer's body for the acquisition of physiological data as described herein. For example, the strap 102 may include a slim elastic band formed of any suitable elastic material such as rubber or a woven polymer fiber such as a woven polyester, polypropylene, nylon, spandex, and so forth. The strap 102 may be adjustable to accommodate different wrist sizes, and may include any latches, hasps, or the like to secure the wearable monitor 104 in an intended position for monitoring a physiological signal. While a wrist-worn device is depicted, it will be understood that the wearable monitor 104 may be configured for positioning in any suitable location on a user's body, based on the sensing modality and the nature of the signal to be acquired. For example, the wearable monitor 104 may be configured for use on a wrist, a forearm, an ankle, a lower leg, a bicep, a chest, side torso, back, a gluteus, behind the ear, forehead, or any other suitable location(s), and the strap 102 may be, or may include, a waistband or other elastic band or the like within an article of clothing or accessory. In another aspect, the wearable monitor 104 may be configured as a ring, earring, stick-on, clip-on, head-mounted (e.g., glasses or goggles), or other article of clothing or accessory that can be worn by a user, and that contains suitable instrumentation, memory, and/or processing for physiological monitoring as described herein. The wearable monitor 104 may also or instead be structurally configured for placement on or within a garment, e.g., permanently or in a removable and replaceable manner. To that end, the wearable monitor 104 may be shaped and sized for placement within a pocket, slot, and/or other housing that is coupled to or embedded within a garment. In such configurations, the pocket or other retaining arrangement on the garment may include sensing windows or the like so that the wearable monitor 104 can operate while placed for use in the garment. U.S. Pat. No. 11,185,292 and U.S. Pat. Pub. No. 2024/0106283 describe non-limiting example embodiments of suitable wearable monitors 104, and are incorporated herein by reference in their entirety. And while the present disclosure may refer to a wrist-worn wearable or other wearable, it should be understood that any of the other locations or forms described herein are also included unless expressly stated to the contrary or otherwise clear from the context.

The system 100 may include any hardware components, subsystems, and the like to support various functions of the wearable monitor 104, such as data collection, processing, display, and communications with external resources. For example, the system 100 may include hardware for a heart rate monitor using, e.g., photoplethysmography, electrocardiography, or any other technique(s). The system 100 may be configured such that, when the wearable monitor 104 is placed for use about a wrist (or at some other body location), the system 100 initiates acquisition of physiological data from the wearer. In some embodiments, the pulse or heart rate may be acquired optically based on a light source (such as light-emitting diodes (LEDs)) and optical detectors in the wearable monitor 104. The LEDs may be positioned to direct illumination toward the user's skin, and optical detectors such as photodiodes may be used to capture illumination intensity measurements indicative of illumination from the LEDs that is reflected and/or transmitted by or through the wearer's skin, or depending on the configuration, through capillaries or arteries.

The system 100 may be configured to record other physiological and/or biomechanical parameters, including, but not limited to, skin temperature (using a thermometer), galvanic skin response (using a galvanic skin response sensor), motion (using one or more multi-axis accelerometers and/or gyroscopes), blood pressure (via physical pressure measurements or other means), sound, electrocardiograms, and the like, as well as environmental or contextual parameters such as ambient light, ambient temperature, humidity, time of day, location, and so forth. For example, the wearable monitor 104 may include sensors such as accelerometers and/or gyroscopes for motion detection, sensors for environmental temperature sensing, sensors to measure electrodermal activity (EDA), sensors to measure galvanic skin response (GSR) sensing, and so forth. The system 100 may also or instead include other systems or subsystems supporting additional functions of the wearable monitor 104. For example, the system 100 may include communications systems to support, e.g., near-field communications, proximity sensing, touch sensing (e.g., via capacitive or resistive sensors), Bluetooth communications, Wi-Fi communications, cellular communications, satellite communications, and so forth. The wearable monitor 104 may also or instead include components such as a Global Positioning System (GPS), a display and/or user interface, a clock and/or timer, and so forth.

The wearable monitor 104 may include one or more sources of battery power, such as a first battery within the wearable monitor 104 and a second battery 106 that is removable from and replaceable to the wearable monitor 104 in order to recharge the battery in the wearable monitor 104. The wearable monitor 104 may also or instead include systems for energy harvesting via, e.g., kinetic energy capture, ambient electromagnetic radiation capture, solar/optical energy capture, and so forth, as well as systems for short and/or medium-range wireless energy transfer to receive power from nearby wireless power sources. Also or instead, the system 100 may include a plurality of wearable monitors 104 (and/or other physiological monitors) that can share battery power or provide power to one another, e.g., using a garment power infrastructure, wireless power sharing network, or the like. The system 100 may perform numerous functions related to continuous monitoring, such as automatically detecting when the user is asleep, awake, exercising, and so forth, and such detections may be performed locally at the wearable monitor 104 or at a remote service such as a mobile device or cloud computing resource coupled in a communicating relationship with the wearable monitor 104 and receiving data therefrom. In general, the system 100 may support continuous, independent monitoring of a physiological signal such as a heart rate, and the underlying acquired data may be stored on the wearable monitor 104 for an extended period until it can be uploaded to a remote processing resource for more computationally complex analysis. In one aspect, the wearable monitor 104 may be a wrist-worn photoplethysmography device, although other form factors are also or instead possible as described herein, such as a ring, a bicep band, a calf band, an elastic band in a garment, a patch, a clip-on device, and so forth.

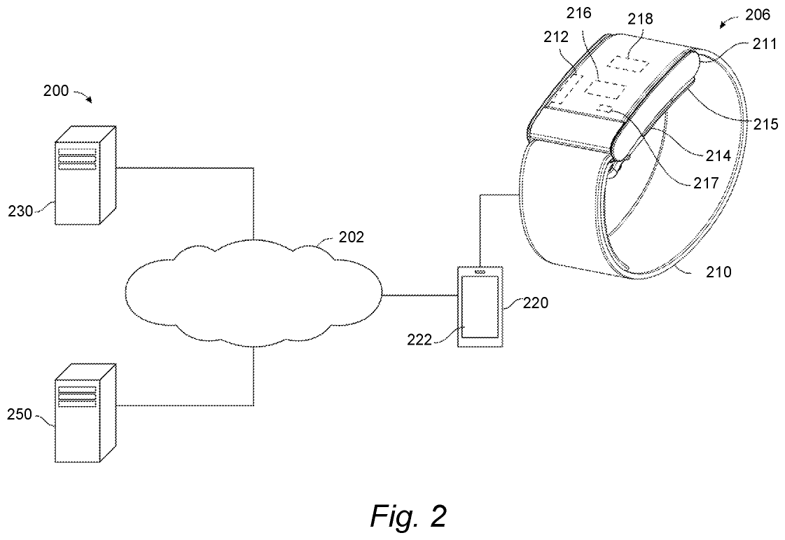

FIG. 2 illustrates a physiological monitoring system. More specifically, FIG. 2 illustrates a physiological monitoring system 200 that may be used with any of the methods or devices described herein. In general, the system 200 may include a physiological monitor 206, a user device 220, a remote server 230 with a remote data processing resource (such as any of the processors or processing resources described herein), and one or more other resources 250, all of which may be interconnected through a data network 202.

The data network 202 may be any of the data networks described herein. For example, the data network 202 may be any network(s) or internetwork(s) suitable for communicating data and information among participants in the system 200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 200. This may also include local or short-range communications infrastructure suitable, e.g., for coupling the physiological monitor 206 to the user device 220, or otherwise supporting communication with local resources. By way of non-limiting examples, short-range communications may include Wi-Fi communications, Bluetooth communications, infrared communications, near-field communications, communications with RFID tags or readers, and so forth.

The physiological monitor 206 may, in general, be any physiological monitoring device or system, such as any of the wearable monitors or other monitoring devices or systems described herein. In one aspect, the physiological monitor 206 may be a wearable physiological monitor shaped and sized to be worn on a wrist or other body location. The physiological monitor 206 may include a wearable housing 211, a network interface 212, one or more sensors 214, one or more light sources 215, a processor 216, a haptic device 217 or other user input/output hardware, a memory 218, and a strap 210 for retaining the physiological monitor 206 in a desired location on a user. In one aspect, the physiological monitor 206 may be configured to acquire heart rate data and/or other physiological data from a wearer in an intermittent or substantially continuous manner. In another aspect, the physiological monitor 206 may be configured to support extended, continuous acquisition of physiological data, e.g., for several days, a week, or more.

The network interface 212 of the physiological monitor 206 may be configured to couple the physiological monitor 206 to one or more other components of the system 200 in a communicating relationship, either directly, e.g., through a cellular data connection or the like, or indirectly through a short-range wireless communications channel coupling the physiological monitor 206 locally to a wireless access point, router, computer, laptop, tablet, cellular phone, or other device that can locally process data, and/or relay data from the physiological monitor 206 to the remote server 230 or other resource(s) 250 as necessary or helpful for acquiring and processing data from the physiological monitor 206. The network interface 212 may also or instead facilitate connections among multiple wearable devices, power sources, and the like, e.g., in a wearable device area network or other multi-device monitoring infrastructure.

The one or more sensors 214 may include any of the sensors described herein, or any other sensors or sub-systems suitable for physiological monitoring or supporting functions. By way of example and not limitation, the one or more sensors 214 may include one or more of a light source (including, e.g., LEDs or other wavelength-specific sources of green light, red light, infrared light, and so forth, as well as broadband illumination), an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, and so forth. The one or more sensors 214 may also or instead include sensors (and accompanying hardware/software) for, e.g., a Global Positioning System, a proximity sensor, an RFID tag reader, an RFID tag, a temporal sensor, an electrodermal activity sensor, an electrocardiogram, a pressure sensor, an acoustic sensor (e.g., a microphone), a camera (e.g., visible light and/or infrared), and the like. The one or more sensors 214 may be disposed in the wearable housing 211, or otherwise positioned and configured for physiological monitoring or other functions described herein. In one aspect, the one or more sensors 214 include a light detector configured to provide light intensity data to the processor 216 (or to the remote server 230) for calculating a heart rate and a heart rate variability. The one or more sensors 214 may also or instead include an accelerometer, gyroscope, and the like configured to provide motion data to the processor 216, e.g., for detecting activities such as a sleep state, a resting state, a waking event, exercise, and/or other user activity. In an implementation, the one or more sensors 214 may include a sensor to measure a galvanic skin response of the user. The one or more sensors 214 may also or instead include electrodes or the like for capturing electronic signals, e.g., to obtain an electrocardiogram and/or other electrically-derived physiological measurements.

The processor 216 and memory 218 may be any of the processors and memories described herein. In one aspect, the memory 218 may store physiological data obtained by monitoring a user with the one or more sensors 214, and/or any other sensor data, program data, or other data useful for operation of the physiological monitor 206 or other components of the system 200. It will be understood that, while only the memory 218 on the physiological monitor is illustrated, any other device(s) or components of the system 200 may also or instead include a memory to store program instructions, raw data, processed data, user inputs, and so forth. In one aspect, the processor 216 of the physiological monitor 206 may be configured to obtain heart rate data from the user, such as heart rate data including or based on the raw data from the sensors 214. The processor 216 may also or instead be configured to determine, or assist in a determination of, a condition of the user related to, e.g., health, fitness, strain, recovery, sleep, or any of the other conditions described herein.

The one or more light sources 215 may be coupled to the wearable housing 211 and controlled by the processor 216. At least one of the light sources 215 may be directed toward the skin of a user adjacent to the wearable housing 211. Light from the light source 215, or more generally, light at one or more wavelengths of the light source 215, may be detected by one or more of the sensors 214, and processed by the processor 216 as described herein.

The system 200 may further include a remote data processing resource executing on a remote server 230. The remote data processing resource may include any of the processors and related hardware described herein, and may be configured to receive data transmitted from the memory 218 of the physiological monitor 206, and to process the data to detect or infer physiological signals of interest such as heart rate, heart rate variability, respiratory rate, pulse oxygen, blood pressure, and so forth. The remote server 230 may also or instead evaluate a condition of the user such as a recovery state, sleep state, exercise activity, exercise type, sleep quality, daily activity strain, and any other health or fitness conditions that might be detected based on such data.

The system 200 may include one or more user devices 220, which may work together with the physiological monitor 206, e.g., to provide a display, or more generally, user input/output, for user data and analysis, and/or to provide a communications bridge from the network interface 212 of the physiological monitor 206 to the data network 202 and the remote server 230. For example, the physiological monitor 206 may communicate locally with a user device 220, such as a smartphone of a user, via short-range communications, e.g., Bluetooth, or the like, for the exchange of data between the physiological monitor 206 and the user device 220, and the user device 220 may in turn communicate with the remote server 230 via the data network 202 in order to forward data from the physiological monitor 206 and to receive analysis and results from the remote server 230 for presentation to the user. In one aspect, the user device(s) 220 may support physiological monitoring by processing or pre-processing data from the physiological monitor 206 to support extraction of heart rate or heart rate variability data from raw data obtained by the physiological monitor 206. In another aspect, computationally intensive processing may advantageously be performed at the remote server 230, which may have greater memory capabilities and processing power than the physiological monitor 206 and/or the user device 220.

The user device 220 may include any suitable computing device(s) including, without limitation, a smartphone, a desktop computer, a laptop computer, a network computer, a tablet, a mobile device, a portable digital assistant, a cellular phone, a portable media or entertainment device, or any other computing devices described herein, including, e.g., supplemental wearable devices and/or computers. The user device 220 may provide a user interface 222 for access to data and analysis by a user, and/or to support user control of operation of the physiological monitor 206. The user interface 222 may be maintained by one or more applications executing locally on the user device 220, or the user interface 222 may be remotely served and presented on the user device 220, e.g., from the remote server 230 or the one or more other resources 250.

In general, the remote server 230 may include data storage, a network interface, and/or other processing circuitry. The remote server 230 may process data from the physiological monitor 206 and perform physiological and/or health monitoring/analyses or any of the other analyses described herein, (e.g., analyzing sleep, determining strain, assessing recovery, and so on), and may host a user interface for remote access to this data, e.g., from the user device 220. The remote server 230 may include a web server or other programmatic front end that facilitates web-based access by the user devices 220 or the physiological monitor 206 to the capabilities of the remote server 230 or other components of the system 200.

The system 200 may include other resources 250, such as any resources that can be usefully employed in the devices, systems, and methods as described herein. For example, these other resources 250 may include other data networks, databases, processing resources, cloud data storage, data mining tools, computational tools, data monitoring tools, algorithms, and so forth. In another aspect, the other resources 250 may include one or more administrative or programmatic interfaces for human actors such as programmers, researchers, annotators, editors, analysts, coaches, and so forth, to interact with any of the foregoing. The other resources 250 may also or instead include any other software or hardware resources that may be usefully employed in the networked applications as contemplated herein. For example, the other resources 250 may include payment processing servers or platforms used to authorize payment for access, content, or option/feature purchases. In another aspect, the other resources 250 may include certificate servers or other security resources for third-party verification of identity, encryption or decryption of data, and so forth. In another aspect, the other resources 250 may include a desktop computer or the like co-located (e.g., on the same local area network with, or directly coupled to through a serial or USB cable) with a user device 220, wearable strap 210, or remote server 230. In this case, the other resources 250 may provide supplemental functions for components of the system 200 such as firmware upgrades, user interfaces, and storage and/or pre-processing of data from the physiological monitor 206 before transmission to the remote server 230.

The other resources 250 may also or instead include one or more web servers that provide web-based access to and from any of the other participants in the system 200. While depicted as a separate network entity, it will be readily appreciated that the other resources 250 (e.g., a web server) may also or instead be logically and/or physically associated with one of the other devices described herein, and may, for example, include or provide a user interface 222 for web access to the remote server 230 or a database or other resource(s) to facilitate user interaction through the data network 202, e.g., from the physiological monitor 206 or the user device 220.

In another aspect, the other resources 250 may include fitness equipment or other fitness infrastructure. For example, a strength training machine may automatically record repetitions and/or added weight during repetitions, which may be wirelessly accessible by the physiological monitor 206 or some other user device 220. More generally, a gym may be configured to track user movement from machine to machine, and report activity from each machine in order to track various strength training activities in a workout. The other resources 250 may also or instead include other monitoring equipment or infrastructure. For example, the system 200 may include one or more cameras to track motion of free weights and/or the body position of the user during repetitions of a strength training activity or the like, and/or the cameras may be integrated into the physiological monitor 206 or other user device 220. Similarly, a user may wear, or have embedded in clothing, tracking fiducials such as visually distinguishable objects for image-based tracking, or radio beacons or the like for other tracking. In another aspect, weights may themselves be instrumented, e.g., with sensors to record and communicate detected motion, and/or beacons or the like to self-identify type, weight, and so forth, in order to facilitate automated detection and tracking of exercise activity with other connected devices.

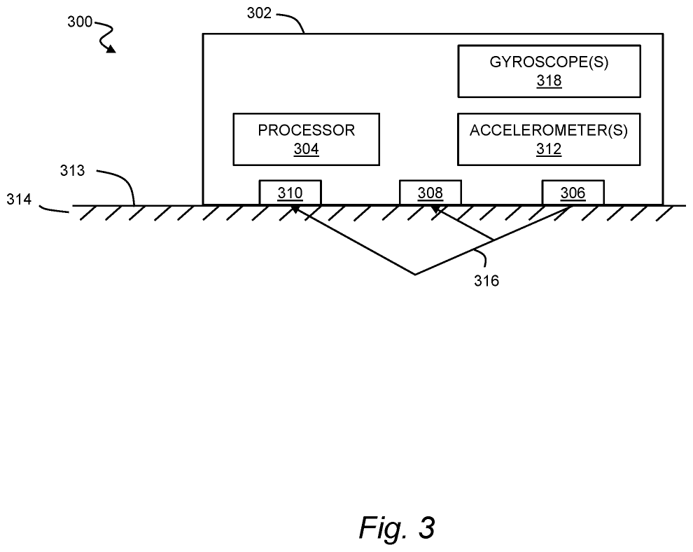

FIG. 3 shows a sensing system. In general, the system 300 may include a physiological monitor 302 with a processor 304, a light source 306, a first sensor 308 (e.g., a first photodetector), a second sensor 310 (e.g., a second photodetector), one or more accelerometers 312, one or more gyroscopes 318, and any other hardware or other components and systems suitable for physiological monitoring as described herein. The physiological monitor 302 may be positioned for use against a surface 313 of the skin 314 of a user where the light source 306 and sensors 308, 310 can contact the skin 314 for acquisition of physiological data. Although not depicted, it will be understood that the physiological monitor 302 may generally be retained in position using any of the straps, garments, patches, bands, clamps, clips, or the like described herein, and/or integrated into other wearable garments, accessories, and the like, such as audio earbuds, earrings or similar, glasses and/or other eyewear, a ring, a headband, and so forth.

The processor 304 may be any microprocessor, microcontroller, application-specific integrated circuit, or other processing circuitry or combination of the foregoing suitable for controlling operation of the physiological monitor and acquiring physiological data.

The light source 306 may include one or more light-emitting diodes or other sources of illumination, and may be positioned within the physiological monitor 302 such that, when the physiological monitor 302 is placed for use on the skin 314, the light source 306 directs illumination toward the skin 314 and the illumination is reflected back toward the sensors 308, 310 as indicated by arrows 316 (or transmitted through the tissue to one or more opposing sensors), where the intensity can be measured. In one aspect, the light source 306 may include light-emitting diodes that emit light in the green, red, infrared, near-infrared, or other suitable wavelength ranges, which can provide desired light transmission through human skin, facilitating low-power transmission of measurable illumination to the sensors 308, 310, although other illumination sources and wavelengths may also or instead be used.

The sensors 308, 310 may be oriented to contact the skin 314 when the physiological monitor 302 is placed for use on this skin 314, and positioned so that the sensors 308, 310 can capture illumination reflected and/or transmitted by the skin from the light source 306. In general, the sensors 308, 310 may include photodiodes, photodetectors, or any other sensor(s) responsive to illumination from the light source 306. This may include broadband optical sensors, narrowband optical sensors, filtered sensors, or the like. In general, a first sensor 308 may be positioned closer to the light source 306 than a second sensor 310 to facilitate detection of differential intensity in the measured wavelength(s). For example, the first sensor 308 may be positioned 1-4 millimeters from the light source 306 and the second sensor 310 may be positioned 2-8 millimeters from the light source, or about twice as far as the first sensor 308 from the light source 306.

Other spacings may also or instead be used depending on, e.g., the intensity of the light source 306, the sensitivity of the sensors 308, 310, the contact force of the physiological monitor 302 on the skin 314, the degree of incursion of ambient light, the physiological measurements/properties of interest, and so forth. In one aspect, the sensors 308, 310 may be linearly arranged in a straight line away from the light source 306. While this provides consistency in comparative measurements, it is not strictly required, and the sensors may be displaced in any of a number of directions away from the light source 306 provided they both contact the skin 314 in a manner that permits capture of light through the skin 314 from the light source 306. In another aspect, the physiological monitor 302 may include one or more other light sources and/or light sensors, which may be arranged to improve accuracy and/or provide redundancy for the contact detection, or to support other measurements such as oxygenation or skin thickness. This may include light sources/sensors using different ranges of wavelengths, different patterns of illumination, and so forth. In another aspect, two sensors may be positioned at different distances from a perimeter of the physiological monitor 302 so that the sensors can acquire differential intensity values for ambient light incident on the skin and transmitted through the skin to the sensors.

In operation, the processor 304 may acquire raw intensity data from the sensors 308, 310, and perform local calculations such as pre-processing raw data for heart rate measurements, or evaluating whether the physiological monitor 302 is properly placed for use on the skin 314.

The accelerometer 312 may include, e.g., one or more single-axis or multi-axis accelerometers, which may usefully measure motion of the physiological monitor 302 to support calculations such as automated activity detection, device on/off evaluation, degree of musculoskeletal activation, and so forth. Other motion and orientation sensing hardware—such as one or more gyroscopes 318, inertial motion sensors, and/or other micro-electromechanical system (MEMS) sensors—may also or instead be used for these purposes. More generally, the physiological monitor 302 may include any additional components, subsystems, and the like suitable for supporting various modes of physiological monitoring and contextual data acquisition as described herein.

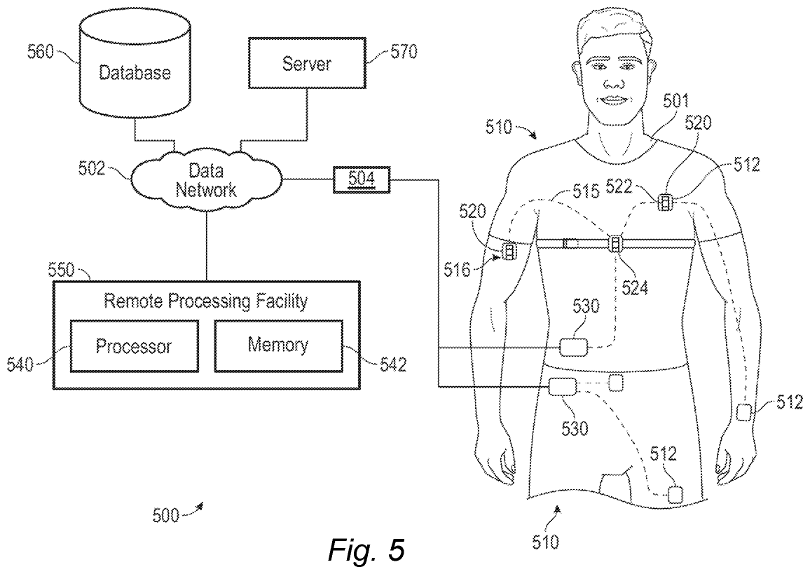

The physiological monitors described herein—e.g., in the systems 100, 200, 300 described above or elsewhere herein—may be provided in one or more different form factors. That is, although a wrist-worn device is illustrated in FIGS. 1 and 2, and garments with sensors are illustrated in FIG. 5, other form factors are also or instead possible, some of which are discussed below by way of example.

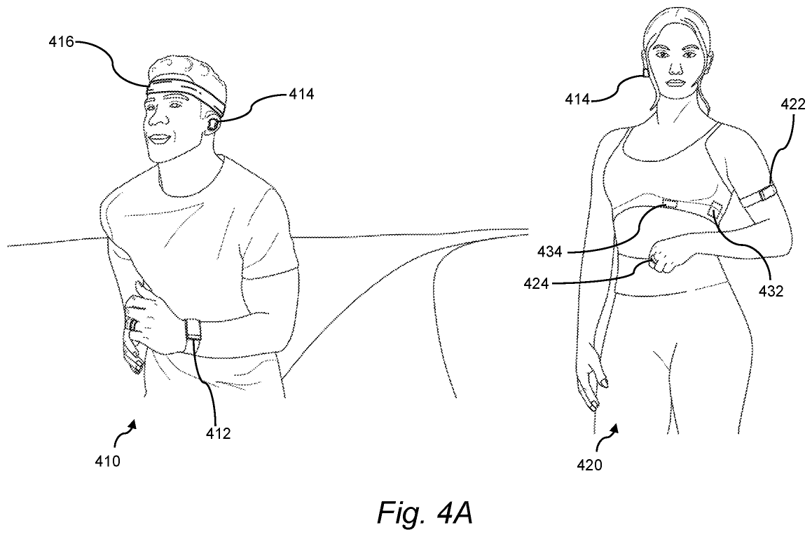

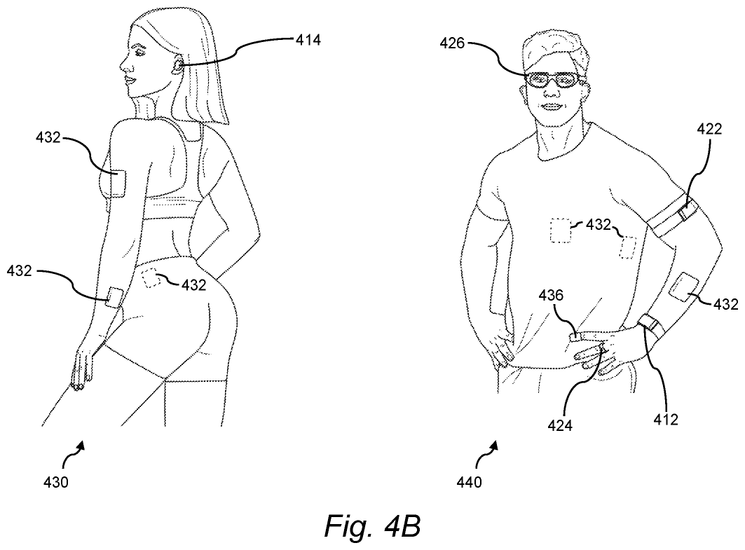

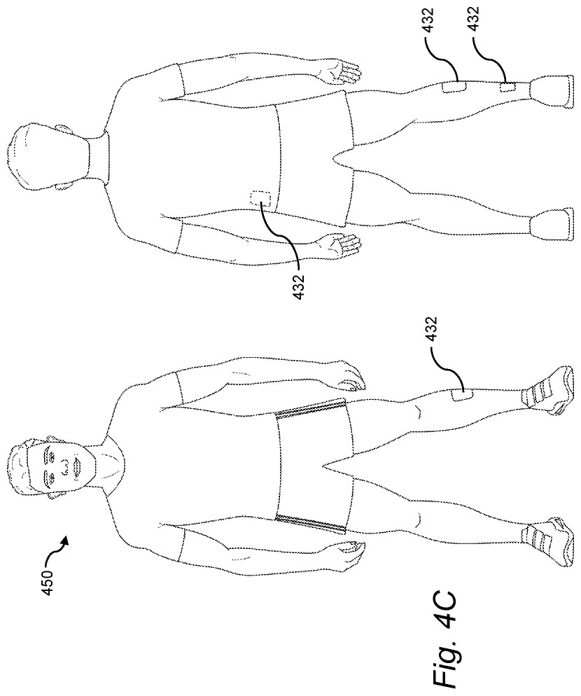

FIGS. 4A-4C illustrate physiological monitoring devices. The illustrated devices may include any of the hardware, software, and/or other components described herein for physiological sensing and/or other functions, and may be embodied in various form factors for various use cases. These various form factors may be used individually or as multiple independent or cooperating physiological monitoring devices, and may include two or more devices of the same type (e.g., two wrist-worn devices, two or more patches, and so on), and/or two or more different types of devices. Moreover, other form factors, and combinations thereof, may also or instead be used for physiological monitoring as described herein. It will further be understood that each of the different example form factors shown in these figures or elsewhere herein may include any one or more of the various sensors, emitters, processors, memories, interfaces, power supplies, and/or other processing and control circuitry, including without limitation any of the foregoing described herein, e.g., with reference to FIGS. 1-3 above.

FIG. 4A shows a first user 410 and a second user 420. The first user 410 may be wearing one or more physiological monitors, such as a wrist-worn device 412 (such as any described herein), an ear-worn device 414 (including on-ear devices retained with a clamp, clip, or other mechanism, and/or in-ear devices, such as earbuds or the like, that are retained at least in part within the ear canal), and a headband 416 or similar.

In one aspect, an ear-worn device 414 may be structurally configured to be partially or entirely inserted within an ear canal of the first user 410. In another aspect, the ear-worn device 414 may be configured to be worn on the ear lobe, or in some other location on the ear where, e.g., temperature, blood flow, respiration, and/or other physiological parameters can be measured. In one aspect, an ear-worn device 414 may be configured for heart rate monitoring, such as any of the heart rate monitoring described herein. For example, this may include continuous heart rate monitoring with optical sensors based on changes in blood volume beneath the skin. The ear-worn device 414 may also or instead be configured for temperature monitoring. For example, the ear-worn device 414 may include one or more infrared sensors, thermistors, thermocouples, or the like to measure the temperature of the ear canal and/or other surfaces. Surface measurements may also or instead be used to support other inferences about body temperature, heat dissipation, and the like, which may be related to current activity levels, general health, and wellness, and so forth.

In another aspect, the ear-worn device 414, or any of the other devices described herein, may be configured for activity tracking. For example, the ear-worn device 414 may include one or more accelerometers, gyroscopes, Global Positioning System (GPS) sensors, and so forth to detect motion and provide information about physical activity levels. This may, for example, include large-scale motion, such as geographical movement and elevation changes that can be tracked with GPS or the like, or local movement detected by the ear-worn device 414, which may be tracked with multi-axis gyroscopes, multi-axis accelerometers, and so forth. These latter sensors may be used to infer, e.g., steps taken, gait analysis, activity type, activity level, and/or overall movement.

The ear-worn device 414, or any of the other devices described herein, may also or instead be configured for blood pressure monitoring. This may, for example, include techniques based on cardiovascular waveform analysis (e.g., using the shape of a PPG or ECG signal from a single location), pulse transit time (e.g., based on the time difference between waveforms at two or more physical locations on the body with two or more monitors), pulse wave velocity (similar to pulse transit time, but over longer arterial distances), physical pulse monitoring (e.g., with pressure sensors, haptic stimulus responses, or other mechanical and/or dynamic techniques), tonometry (measuring the force required to counteract arterial pressure), oscillometric measurement (measuring oscillations in the arterial wall as a cuff deflates around a region of interest), volume clamping (measuring changes in pressure that are required to maintain constant blood volume in a region of interest), and so forth. Some of these blood pressure monitoring techniques are better suited to specific types and locations of monitors and may be more suited to, e.g., wristbands, bicep bands, chest straps, finger rings, and so forth, but are included here for completeness.

The ear-worn device 414, or any of the other devices described herein, may also or instead be configured for electrodermal activity (EDA) monitoring. For example, the ear-worn device 414 may include one or more electrodes in contact with the skin, which may be used to measure the electrical conductance thereof, and to infer, e.g., sweat levels, skin hydration, and/or other parameters correlated to skin conductance. Electrodes may also or instead be used for, e.g., ECG monitoring or the like.

The ear-worn device 414, or any of the other devices described herein, may also or instead be configured to sense blood oxygen saturation (also referred to as pulse oximetry or SpO2) monitoring. To this end, the ear-worn device 414 may include one or more optical sources and detectors, and the system may use different absorption spectra of oxygenated and deoxygenated hemoglobin to estimate pulse oxygen saturation. In another aspect, the ear-worn device 414, or any of the other devices described herein, may be configured for brainwave monitoring, e.g., using electroencephalogram (EEG) sensors to monitor brainwave activity.

The ear-worn device 414, or any of the other devices described herein, may also or instead be configured for respiration rate monitoring. In one aspect, respiration rate may be inferred using respiratory sinus arrhythmia or other techniques to infer respiration rate from a measured heart rate signal over time. In another aspect, respiration rate may be inferred from physical changes in the ear canal (or chest, or other body part, where applicable to a particular sensor). Other techniques may also or instead be used. For example, the ear-worn device 414 may include a microphone or other audio transducer, and the respiration rate may be inferred from audio data acquired from the user.

In another aspect, a headband 416 may be structurally and programmatically configured for physiological sensing and/or monitoring using any of the systems and methods described herein. For example, the headband 416 may be configured to monitor heart rate, temperature, brain activity, electromyography, galvanic skin response, motion, activity, and so forth. In general, the sensors and processing may be adapted for the form factor of the headband 416. For example, the headband 416 may use temperature sensors to measure skin temperature and/or ambient temperature around the head. For brain activity, the headband 416 may include EEG sensors or the like embedded within the headband 416 to measure electrical activity in the brain, which can be used for monitoring brain waves associated with different states such as relaxation, concentration, and/or sleep. More generally, any physiological monitoring techniques described herein that can be adapted for use in a corresponding form factor may be deployed, either alone or in combination, for physiological monitoring with the headband 416. In another aspect, the headband 416 may incorporate a brain-computer interface (BCI) for control of a physiological monitoring system. This may, for example, include any system suitable for direct communication between the brain and external devices based on, e.g., signal acquisition using techniques such as electroencephalography, processing of these raw signals, feature extraction and translation, and then command execution based on an inferred user intention.

The second user 420 may be wearing one or more physiological monitors such as an ear-worn device 414 (which may be any as described herein, and which may be configured as a clamp, clip, earring, or similar, as shown), a bicep band 422, a ring 424, a patch 432 (such as any as described herein, e.g., with reference to FIG. 4B), and a band sensor 434.

The bicep band 422 may be configured for physiological monitoring and sensing using any of the systems and methods described herein, e.g., by retaining a sensor in place with the bicep band 422 or integrating components of the sensor into the bicep band 422, or some combination of these. The bicep band 422 may be configured to monitor heart rate, motion, activity, temperature, blood pressure, blood oxygen saturation, hydration, body composition, ultraviolet light exposure, electrodermal activity, and so forth, as well as combinations of the foregoing. In one aspect, electromyography (EMG) may be used to measure electrical activity in the muscles, e.g., with one or more electrical contacts or the like embedded in the bicep band 422, which can provide information about muscle contraction and fatigue during physical activity. Body composition analysis may be performed using, e.g., bioelectrical impedance analysis to estimate various components of body composition such as fat (percentage or mass), muscle (percentage or mass), and hydration. In another aspect, the bicep band 422 may include one or more sensors to measure ambient light, and more specifically, ambient ultraviolet (UV) light. This may be used to monitor UV exposure, and to provide recommendations to the user to meet certain healthy thresholds for, e.g., vitamin D synthesis, mood, and immune function, and/or to provide alerts concerning possible overexposure. In another aspect, the bicep band 422 or other form factors described herein may be adapted for gesture control based on the capture of motion signals and corresponding inferences of user intent. While a bicep band 422 is illustrated, it will be understood that similar bands for other body parts may also or instead be used, such as leg bands (or more specifically, thigh bands, calf bands, ankle bands, etc.), chest bands, abdomen bands, neck bands, wristbands, and so forth.

The ring 424 may be configured for physiological monitoring and sensing using any of the systems and methods described herein. For example, the ring 424 may be configured to monitor heart rate, motion, activity, sleep, temperature, blood pressure, respiration rate, blood oxygen saturation, hydration, UV exposure, and so forth. A ring 424 is also advantageously positioned to capture a wide range of hand motions, and may be configured for gesture control of physiological monitoring and/or related hardware and software. The ring 424 may be configured for wearing on a finger, as shown in the figure, or another portion of a wearer's body (e.g., a thumb, a toe, and so forth).

The band sensor 434 may be the same or similar to the other monitors described herein and/or any of the bands as described herein. In an aspect, the band sensor 434 may include a monitor inserted into (e.g., placed into a pocket or the like), coupled with, embedded within, or the like, a strap or band, e.g., an elastic band in an article of clothing, an accessory, or similar.

FIG. 4B shows a third user 430 and a fourth user 440. The third user 430 may be wearing one or more physiological monitors such as an ear-worn device 414, which may be the same as or similar to any of those described herein, and one or more patches 432 that include sensors and the like to support physiological monitoring. By way of example, a patch 432 may be configured for physiological monitoring and sensing of heart rate monitoring, temperature, activity, motion, blood pressure, blood oxygen saturation, respiration rate, blood glucose, perspiration, hydration, ultraviolet exposure, and so forth, as well as combinations of the foregoing. In one aspect, the patch 432 may include a continuous glucose monitor with a sensor for insertion into fatty tissue under the skin, along with a transmitter to wirelessly transmit glucose data to a smartphone or other device. In another aspect, the patch 432 may include a hydration monitor using, e.g., electrical impedance analysis to measure resistance and reactance of body tissue with a small electrical current, or bioimpedance spectroscopy to measure impedance at various frequencies of electrical current. Hydration monitoring may also or instead use a wearable patch to collect sweat and analyze electrolyte concentrations correlated to hydration. Other techniques for measuring hydration using, e.g., near-infrared spectroscopy or capacitance hygrometry, may also or instead be employed where suitable adaptations can be made to any of the wearable monitors described herein. In another aspect, the patch 432, or any of the other monitors described herein, may be adapted to monitor environmental conditions such as temperature, humidity, air quality, noise, light, and the like that might be used to supplement physiological monitoring when evaluating the condition of a user. In another aspect, the patch 432, or any of the other monitors described herein, may be adapted for electrodermal activity monitoring, e.g., for tracking autonomic nervous system activity, stress, and the like based on galvanic skin response. One or more patches 432 may be coupled to a user in one or more of a plurality of locations on the body, such as those shown on the third user 430—e.g., a portion of an arm (e.g., the upper arm and/or the lower arm), and on or near the gluteus maximus, and similar. Other locations are also or instead possible, such as the chest, the abdomen, the forehead or temples, the wrist, a hand, a finger, a foot, a neck, a backside, the pelvic region, a portion of the back, a portion of a leg, and so forth.

The fourth user 440 may be wearing one or more physiological monitors such as a bicep band 422, a wrist-worn device 412, a ring 424, and a patch 432, which may be the same or similar to any of the monitors described herein. The fourth user 440 further is shown with eyewear 426 and a fingertip monitor 436, as further explained below by way of example.

The eyewear 426 may include sensors or the like in contact areas or similar, such as a temple region, face region (e.g., via the frame or lens), or other head portion of the fourth user 440. For example, the eyewear 426 may be configured for physiological monitoring and sensing of heart rate, temperature, brain activity, motion, activity type, blood pressure, blood oxygen saturation, and so forth, as well as combinations of the foregoing. In one aspect, the eyewear 426 may employ electrooculography (EOG) to measure electrical activity of the muscles around the eyes or another region of the head/face, which can be used, e.g., to track eye movements and provide insights into cognitive states, attention levels, fatigue, and so forth. In another aspect, one or more EEG sensors may be integrated into the frame and/or temples of the eyewear 426 to measure electrical activity in the brain. The eyewear 426 may also or instead be configured to perform eye tracking using cameras and/or infrared or other sensors to monitor movement of the eyes, which can be used for various applications, including human-computer interaction, attention monitoring, and so forth. The eyewear 426 may also or instead be configured for augmented reality (AR) and virtual reality (VR) biometrics, e.g., where the eyewear 426 can include sensors that monitor physiological parameters to enhance user experience and safety, and to visually present information to the user related to any of the foregoing. In another aspect, the eyewear 426 may include cameras, microphones, and the like for recording and tracking environment information.

The fingertip monitor 436 may include a clamp, clip, or the like, and may be the same or similar to any of the physiological monitors described herein. In some aspects, the fingertip monitor 436 may include a pulse oximeter configured to measure oxygen saturation and/or heart rate for monitoring respiratory and/or cardiovascular health.

FIG. 4C shows the front and back of a fifth user 450 showing further example locations for a patch 432 or the like as described herein.

More generally, any one or more of the sensing modalities described herein may, provided suitable adaptations can be made, be deployed in any one or more of the wearable devices described herein. Furthermore, one or more of the wearable devices may communicate with one or more other wearable devices and/or with a control device such as a smartphone or other computing device, to perform cooperative monitoring. For example, various monitoring techniques, such as electrocardiography blood pressure measurements using pulse transit time, may usefully be performed by combining signals from sensors at two or more different body locations, and a control device may usefully acquire signals from multiple devices and locations to perform such analysis. Similarly, multiple motion signals from different body locations may be used to refine activity detection, measure body temperature, and so forth. Thus, in one aspect, two or more wearable devices may cooperate with one another to perform an integrated sensing operation such as any of those described herein.

In another aspect, any one or more of the wearable electronic devices described herein may use energy harvesting to generate power from various external sources, and/or to supplement power supplied by an internal battery or the like. For example, a device may use solar energy harvesting to extract solar energy from ambient light sources. This may include integrating solar cells or other ambient light collectors into the wearable device to capture energy from sunlight and/or artificial light sources. In another aspect, the device may use kinetic energy harvesting to generate energy from movements by a user of the device. In another aspect, the device may use thermal energy harvesting to generate power based on differences between the body of the wearer and the surrounding environment. The device may also or instead use vibration energy harvesting, radio frequency energy harvesting (e.g., by capturing ambient RF signals, such as Wi-Fi or cellular signals, and converting them into usable electrical power), ambient light harvesting, and so forth. Other techniques may also or instead be used to provide external power, such as beam steering or resonant techniques for short-range or medium-range radio frequency power transfers. More generally, any technique or combination of techniques for powering a device, and/or for supplementing an internal power source such as a battery, with power from ambient sources may be used to power one of the monitoring devices described herein.

FIG. 5 shows a smart garment system. One limitation on wearable sensors can be body placement. Devices are typically wrist-based, and may occupy a location that a user would prefer to reserve for other devices or jewelry, or that a user would prefer to leave unadorned for aesthetic or functional reasons. This location also places constraints on what measurements can be taken, and may also limit user activities. For example, a user may be prevented from wearing boxing gloves while wearing a sensing device on their wrist. To address this issue, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user's body for supporting monitoring hardware, and may further enable sensing techniques that cannot be achieved with single sensing devices. For example, embedding a plurality of physiological sensors or other electronic/communication devices in a shirt may allow electrical sensors to be placed around a torso to support electrocardiogram (ECG) based heart rate measurements, or placed around muscles such as the pectoralis major, latissimus dorsi, biceps brachii, and other major muscle groups to support muscle oxygen saturation measurements. In another aspect, optical sensors may be positioned along an arterial pathway or the like to support pulse transit time measurements for calculation of blood pressure. The infrastructure provided by a garment may also support other supplemental functions beyond physiological monitoring. For example, wireless antennas may be placed above the upper portion of the thoracic spine to achieve desired communications signals, or a contactless payment system may be embedded in a sleeve cuff for interactions with a payment terminal. Smart garments may also free up body surfaces for other devices. For example, if sensors in a wrist-worn device that provide heart rate monitoring and step counting can be instead embedded in a user's undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment that incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications, and so forth. The garment may also or instead include a power supply, a power management system, processing hardware, data storage, and so forth, any of which may support enriched functions for the smart garment.

In general, the smart garment system 500 illustrated in FIG. 5 may include a plurality of components—e.g., a garment 510, one or more modules 520, a controller 530, a processor 540, a memory 542, and so on—capable of communicating with one another over a data network 502. The garment 510 may be wearable by a user 501 and configured to communicate with a module 520 having a physiological sensor 522 that is structurally configured to sense a physiological parameter of the user 501. As discussed herein, the module 520 may be controllable by the controller 530 based at least in part on a location 516 where the module 520 is located on or within the garment 510. This position-based information may be derived from an interaction and/or communication between the module 520 and the garment 510 using various techniques. It will be understood that, while two controllers 530 are shown, the garment 510 may include a single inter-garment controller, or any number of separate controllers 530 in any number of garments 510 (e.g., one per garment, or one for all garments worn by a person, etc.), and/or controllers may be integrated into other modules 520.

For communication over the data network 502, the system 500 may include a network interface 504, which may be integrated into the garment 510, included in the controller 530, or in some other module or component of the system 500, or some combination of these. The network interface 504 may generally include any combination of hardware and software configured to wirelessly communicate data to remote resources. For example, the network interface 504 may use a local connection to a laptop, smartphone, or the like that couples, in turn, to a wide area network for accessing, e.g., web-based or other network-accessible resources. The network interface 504 may also or instead be configured to couple to a local access point such as a router or wireless access point for connecting to the data network 502. In another aspect, the network interface 504 may be a cellular communications data connection for direct, wireless connection to a cellular network or the like.

The data network 502 may be any as described herein. By way of example, some embodiments of the system 500 may be configured to stream information wirelessly to a social network, a data center, a cloud service, and so forth. In some embodiments, data streamed from the system 500 to the data network 502 may be accessed by the user 501 (or other users) via a website. The network interface 504 may thus be configured such that data collected by the system 500 is streamed wirelessly to a remote processing facility 550, database 560, and/or server 570 for processing and access by the user. In some embodiments, data may be transmitted automatically, without user interaction, for example by storing data locally and transmitting the data over available local area network resources when a local access point such as a wireless access point or a relay device (such as a laptop, tablet, or smartphone) is available. In some embodiments, the system 500 may include a cellular system or other hardware for independently accessing network resources from the garment 510 without requiring local network connectivity. It will be understood that the network interface 504 may include a computing device such as a mobile phone or the like. The network interface 504 may also or instead include or be included on another component of the system 500, or some combination of these. Where battery power or communications resources can advantageously be conserved, the system 500 may preferentially use local networking resources when available, and reserve cellular communications for situations where a data storage capacity of the garment 510 is reaching capacity. Thus, for example, the garment 510 may store data locally up to some predetermined threshold for local data storage, below which data is transmitted over local networks when available. The garment 510 may also transmit data to a central resource using a cellular data network only when local storage of data exceeds the predetermined threshold.

The garment 510 may include one or more designated areas 512 for positioning a module to sense a physiological parameter of the user 501 wearing the garment 510. One or more of the designated areas 512 may be specifically tailored for receiving a module 520 therein or thereon. For example, a designated area 512 may include a pocket structurally configured to receive a module 520 therein. Also or instead, a designated area 512 may include a first fastener configured to cooperate with a second fastener disposed on a module 520. One or more of the first fastener and the second fastener may include at least one of a hook-and-loop fastener, a button, a clamp, a clip, a snap, a projection, and a void.

By placing a pocket or the like in one of these designated areas 512, a position of a module 520 can be controlled, and where an RFID tag, sensor, or the like is used, the designated area 512 can specifically sense when a module 520 is positioned there for monitoring, and can communicate the detected location to any suitable control circuitry.

The garment 510 may also or instead incorporate other infrastructure 515 to cooperate with a module 520. For example, the garment infrastructure 515 may include infrastructure 515 related to ECG devices, such as ECG pads (or otherwise electrically conductive sensor pads and/or electrodes that connect to the module 520, controller 530, and/or another component of the system 500), lead wires, and the like. By way of further example, the garment infrastructure 515 may include wires or the like embedded in the garment 510 to facilitate wired data or power transfer between installed modules 520 and other system components (including other modules 520). The infrastructure 515 may also or instead include integrated features for, e.g., powering modules, supporting data communications among modules, and otherwise supporting operation of the system 500. The infrastructure 515 may also or instead include location or identification tags or hardware, a power supply for powering modules 520 or other hardware, communications infrastructure as described herein, a wired intra-garment network, or supplemental components such as a processor, a Global Positioning System (GPS), a timing device, e.g., for synchronizing signals from multiple garments, a beacon for synchronizing signals among multiple modules 520, and so forth. More generally, any hardware, software, or combination of these suitable for augmenting operation of the garment 510 and a physiological monitoring system using the garment 510 may be incorporated as infrastructure 515 into the garment 510 as contemplated herein.

The modules 520 may generally be sized and shaped for placement on or within one or more designated areas 512 of the garment 510. For example, in certain implementations, one or more of the modules 520 may be permanently affixed on or within the garment 510. In such instances, the modules 520 may be washable. Also or instead, in certain implementations, one or more of the modules 520 may be removable and replaceable relative to the garment 510. In such instances, the modules 520 need not be washable, although a module 520 may be designed to be washable and/or otherwise durable enough to withstand a prolonged period of engagement with a designated area 512 of the garment 510. A module 520 may be capable of being positioned in more than one of the designated areas 512 of the garment 510. That is, one or more of the plurality of modules 520 may be configured to sense data using a physiological sensor 522 in a plurality of designated areas 512 of the garment 510.

A module 520 may include one or more physiological sensors 522 and a communications interface 524 programmed to transmit data from at least one of the physiological sensors 522. For example, the physiological sensors 522 may include one or more of a heart rate monitor (e.g., one or more PPG sensors or the like), an oxygen monitor (e.g., a pulse oximeter), a blood pressure monitor, a thermometer, an accelerometer, a gyroscope, a position sensor, a Global Positioning System, a clock, a galvanic skin response (GSR) sensor, or any other electrical, acoustic, optical, camera, or other sensor or combination of sensors and the like useful for physiological monitoring, environmental monitoring, or other monitoring as described herein. In one aspect, the physiological sensors 522 may include a conductivity sensor or the like used for electromyography, electrocardiography, electroencephalography, or other physiological sensing based on electrical signals. The data received from the physiological sensors 522 may include at least one of heart rate data and/or similar data related to blood flow (e.g., from PPG sensors), muscle oxygen saturation data, temperature data, movement data, position/location data, environmental data, temporal data, blood pressure data, and so on.

Thus, certain embodiments include one or more physiological sensors 522 configured to provide continuous measurements of heart rate using photoplethysmography or the like. The physiological sensor 522 may include one or more light emitters for emitting light at one or more desired frequencies toward the user's skin, and one or more light detectors for receiving light reflected from the user's skin. The light detectors may include a photo-resistor, a phototransistor, a photodiode, and the like. A processor may process optical data from the light detector(s) to calculate a heart rate based on the measured, reflected light. The optical data may be combined with data from one or more motion sensors, e.g., accelerometers and/or gyroscopes, to minimize or eliminate noise in the heart rate signal caused by motion or other artifacts. The physiological sensor 522 may also or instead provide at least one of continuous motion detection, environmental temperature sensing, electrodermal activity (EDA) sensing, galvanic skin response (GSR) sensing, and the like.

The system 500 may include different types of modules 520. For example, a number of different modules 520 may each provide a particular function. Thus, the garment 510 may house one or more of a temperature module, a heart rate/PPG module, a muscle oxygen saturation module, a haptic module, a wireless communication module, or combinations thereof, any of which may be integrated into a single module 520 or deployed in separate modules 520 that can communicate with one another. Some measurements, such as temperature, motion, optical heart rate detection, and the like, may have preferred or fixed locations, and pockets or fixtures within the garment 510 may be adapted to receive specific types of modules 520 at specific locations within the garment 510. For example, motion may preferentially be detected at or near extremities, while heart rate data may preferentially be gathered near major arteries. In another aspect, some measurements, such as temperature, may be measured anywhere, but may preferably be measured at a single location in order to avoid certain calibration issues that might otherwise arise through arbitrary placement.

In another aspect, the system 500 may include two or more modules 520 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, blood pressure, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly, where packet loss, latency, and the like may present challenges to real-time processing.

The communications interface 524 may be any as described herein, for example, including any of the features of the network interface 504 described above.

The controller 530 may be configured, e.g., by computer-executable code or the like, to determine a location of the module 520. This may be based on contextual measurements, such as accelerometer data from the module 520, which may be analyzed by a machine learning model or the like to infer a body position. In another aspect, this may be based on other signals from the module 520. For example, signals from sensors, such as photodiodes, temperature sensors, resistors, capacitors, and the like, may be used alone or in combination to infer a body position. In another aspect, the location may be determined based on the proximity of a module 520 to a proximity sensor, RFID tag, or the like at or near one of the designated areas 512 of the garment 510. Based on the location, the controller 530 may adapt operation of the module 520 for location-specific operation. This may include selecting filters, processing models, physiological signal detections, and the like. It will be understood that operations of the controller 530, which may be any controller, microcontroller, microprocessor, or other processing circuitry, or the like, may be performed in cooperation with another component of the system 500, such as the processor 540 described herein, one or more of the modules 520, or another computing device. It will also be understood that the controller 530 may be located on a local component of the system 500 (e.g., on the garment 510, in a module 520, and so on) or as part of a remote processing facility 550, or some combination of these. Thus, in an aspect, a controller 530 is included in at least one of the plurality of modules 520. And, in another aspect, the controller 530 is a separate component of the garment 510, and serves to integrate functions of the various modules 520 connected thereto. The controller 530 may also or instead be remote relative to each of the plurality of modules 520, or some combination of these.

The controller 530 may be configured to control one or more of (i) sensing performed by a physiological sensor 522 of the module 520 and (ii) processing by the module 520 of the data received from a physiological sensor 522. That is, in certain aspects, the combination of sensors in the module 520 may vary based on where it is intended to be located on a garment 510. In another aspect, processing of data from a module 520 may vary based on where it is located on a garment 510. In this latter aspect, a processing resource such as the controller 530 or some other local or remote processing resource coupled to the module 520 may detect the location and adapt processing of data from the module 520 based on the location. This may, for example, include a selection of different models, algorithms, or parameters for processing sensed data.

In another aspect, this may include selecting from among a variety of different activity recognition models based on the detected location. For example, a variety of different activity recognition models may be developed, such as machine learning models, lookup tables, analytical models, or the like, which may be applied to accelerometer data to detect an activity type. Other motion data, such as gyroscope data, may also or instead be used, and activity recognition processes may also be augmented by other potentially relevant data, such as data from a barometer, magnetometer, GPS, and so forth. This may generally discriminate, e.g., between being asleep, at rest, or in motion, or this may discriminate more finely among different types of athletic activity, such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 510, and then use this detected location to select a suitable model for activity recognition. This technique may similarly be applied to calibration models, physiological signal processing models, and the like, or to otherwise adapt processing of signals from a module 520 based on the location of the module 520. In general, determining a location of a module 520 may include, e.g., receiving a sensed location for the module 520, determining the location based on communications between the module 520 and the garment 510, determining the location based on data received from a physiological sensor 522 of the module 520, and so forth.

Once determined using any of the techniques above, the location of a module 520 may be transmitted for storage and analysis to a remote processing facility 550, a database 560, or the like. That is, in addition to the module 520 using this information locally to configure itself for the location in which it is worn, the module 520 may communicate this information to other modules 520, peripherals, or the cloud. Processing this information in the cloud may help an organization determine if a module 520 has ever been installed on a garment 510, which locations are most used, and how modules 520 perform differently in different locations. These analytics may be useful for many purposes and may, for example, be used to improve the design or use of modules 520 and garments 510, either for a population, for a user type, or for a particular user.

As stated above, the system 500 may further include a processor 540 and a memory 542. In general, the memory 542 may bear computer-executable code configured to be executed by the processor 540 to perform processing of the data received from one or more modules 520. One or more of the processor 540 and the memory 542 may be located on a local component of the system 500 (e.g., the garment 510, a module 520, the controller 530, and the like) or as part of a remote processing facility 550 or the like, as shown in the figure. Thus, in an aspect, one or more of the processor 540 and the memory 542 are included on at least one of the plurality of modules 520. In this manner, processing may be performed on a central module or on each module 520 independently. In another aspect, one or more of the processor 540 and the memory 542 are remote relative to each of the plurality of modules 520. For example, processing may be performed on a connected peripheral device, such as a smartphone, laptop, local computer, or cloud resource.

The processor 540 may be configured to assess the quality of the data received from a physiological sensor 522 of the module 520, or otherwise process data as described herein. The memory 542 may store one or more algorithms, models, and supporting data (e.g., parameters, calibration results, user selections, and so forth) and the like for transforming data received from a physiological sensor 522 of the module 520. In this manner, suitable models, algorithms, tuning parameters, and the like may be selected for use in transforming the data based on the location of the module 520 as determined by the controller 530 and/or processor 540 as described herein.

A database 560 may be located remotely and in communication with the system 500 via the data network 502. The database 560 may store data related to the system 500 such as any discussed herein—e.g., sensed data, processed data, transformed data, metadata, physiological signal processing models and algorithms, personal activity history, and the like. The system 500 may further include one or more servers 570 that host data, provide a user interface, process data, and so forth in order to facilitate use of the modules 520 and garments 510 as described herein.

It will be appreciated that the garment 510, modules 520, and accompanying garment infrastructure and remote networking/processing resources may advantageously be used in combination to improve physiological monitoring and achieve modes of monitoring not previously available.

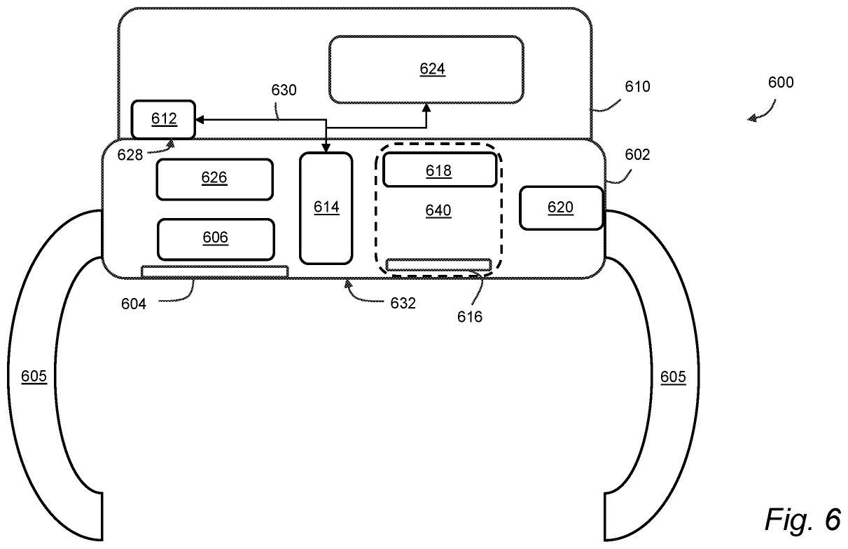

FIG. 6 depicts a functional block diagram of a physiological monitoring device with core temperature sensing. A wearable device 600, such as any of the wearable monitors described herein, may include a housing 602 that contains a biometric sensor 604, such as any of the sensors or sensing circuits described herein, along with a processing circuitry 606 for monitoring the biometric sensor 604. Additionally, the housing 602 may contain a first temperature sensor 616 positioned adjacent to and in thermal contact with the skin of the person wearing the wearable device 600, or otherwise positioned to measure temperature data from the skin. The wearable device 600 may include one or more straps 605 to secure the wearable device 600 in place for use in monitoring as described herein. It will be understood that, while one or more straps 605 are illustrated for securing the wearable device 600 to a user, any band, bracelet, sleeve, compression garment pocket, or other retaining mechanism(s) may also or instead be used. For example, the wearable device 600 may include a ring or similar finger-sized band shaped and sized to slide on and off a finger of a user, the wearable device 600 may be sized to fit into a pocket of a compression garment or the like. The first temperature sensor 616 may, for example, measure temperature based on thermal contact, optical detection, or the like.

The wearable device 600 may include a power supply 610, such as a removable and replaceable power supply or recharging battery. The power supply 610 may include a wireless and/or wired interface for connecting to the wearable device 600, e.g., to support power and data transmissions therebetween. In various embodiments, this permits a controller on the wearable device 600 to controllably activate the power supply 610 for injection of controlled thermal energy into the wearable device 600. Alternatively, where the power supply 610 is configured to control core temperature measurements, the power supply 610 may activate its own charging circuitry or remotely activate internal charging circuitry or other power dissipating circuitry on the wearable device 600 to create an internal, controlled heat source for the wearable device 600. In this latter configuration, the power supply 610 may effectively act as an accessory to the wearable device 600 that adds functionality for core temperature measurement, and the power supply 610 may locally perform calibration, control heat injection, calculate core temperature, and then provide the core temperature in a user-accessible format. For example, in this embodiment, the power supply 610 may store the core temperature, present the core temperature in a display, or transmit the core temperature to another user device or remote processing resource for additional processing.

The power supply 610 may be integrated into the housing 602 as a permanent or removable module or the like. In general, the power supply 610 may include a second temperature sensor 612 that is positioned so that it is adjacent to and thermally coupled to the bottom wall 628 of the power supply 610. In this position, the second temperature sensor 612 can directly physically contact the housing 602 when the power supply 610 is placed on the housing 602 for charging. In embodiments, the second temperature sensor 612 may instead be positioned at a known location between the skin of the user and the heat source of the power supply 610, e.g., in a location that permits measurement of heat flux through the wearable device 600. In embodiments, the housing 602 of the wearable device 600 may also include a controller 614, the first temperature sensor 616, e.g., for measuring a temperature where the housing 602 contacts a wearer's skin, and a third temperature sensor 618, e.g., for measuring a temperature at a location separated from the first temperature sensor 616 (e.g., on an opposing side of the housing, or between the skin contact point and the power supply 610) to permit estimation of heat flux through the wearable device 600, or sub-components thereof.

While the recharging system of a power supply 610, such as a recharging battery, can provide a controllable heat source, the power supply 610 may also or instead include an independently controllable heat source. For example, the power supply 610 may include a Peltier device or other controllable heat source for controlled injection of thermal energy into (or removal of heat from) the system in a manner that permits calibration and core temperature calculation as described herein. Thus, it should be understood that the power supply 610, as described herein, may include a removable recharging battery, but may also or instead include a removable accessory configured to support core temperature measurements in combination with the wearable device 600. In some embodiments, the power supply 610 may omit recharging circuitry, and may instead rely on the Peltier device or some other mechanism as a controlled thermal source, and the power supply 610 may also include control circuitry configured to calibrate the system, obtain thermal measurements, calculate core temperature, and display, store, or transmit core temperature results for use by the user.

The controller 614 may connect to the power supply 610 via an electrical connection 630 including, e.g., a plug, connector, contacts, or antenna (for wireless power and/or data transfer). The electrical connection 630 can also or instead include a bus, one or more wires, a wireless data transceiver, and so forth. In general, the power supply 610 may include a battery pack 624. The battery pack 624 may be a rechargeable battery pack using any battery chemistry and architecture suitable for powering wearable devices such as biometric monitors, smartwatches, or similar consumer electronics.

In one aspect, the controller 614 may include any of the processors or other processing circuitry described herein and may be configured to connect to the power supply 610 to control operation of the battery pack 624 in a first mode of operation under which the battery pack 624 provides power for operating the wearable device 600 and/or charging a battery 626 in the housing 602 that powers the wearable device 600 during use. Further, the controller 614 can operate the power supply 610 to control the battery pack 624 to enter a second mode wherein the power supply 610 acts as a controllable heat source. It will be understood that a separate, controllable heat source may also or instead be used for this purpose, such as one or more resistors for Joule heating, inductive heaters, dielectric heaters, Peltier effect devices, and so forth. However, where the power supply 610 includes a recharging system or other controllable heat dissipating components, this can advantageously support controlled injections of heat into a thermal system without requiring additional heating or power components.

It should also be appreciated that controlled thermal excitation or similar phrases, as used herein, may include any of a variety of thermal stimuli useful for calibration or measurement using the techniques described herein. By way of non-limiting example, thermal excitations may include thermal impulses, thermal impulse trains, thermal step functions, thermal ramps, periodic (e.g., sinusoidal) thermal stimuli, and so forth. In general, provided the thermal excitation can be controlled in a known or measurable manner, the responsive measurements of thermal activity across the wearable device 600 can be used to calibrate and apply a lumped thermal model as described herein. At the same time, while steady-state results provide useful information for characterizing a thermal system, dynamic responses (at single frequencies or multiple frequencies) may also or instead be used, e.g., to characterize frequency response characteristics such as rise time, settling time, time constants, phase shift, attenuation, and so forth in a manner that supports improved system characterization.

Further, while controlled thermal excitation may include a predetermined input or stimulus using a controllable heat source, in some embodiments, core temperature may be estimated transparently. For example, during ordinary use, a battery pack may be coupled to a wearable device for recharging, e.g., as described herein. While recharging, e.g., as orchestrated between the wearable device and a recharging battery, the amount of heat generated by the recharging battery may be measured and used along with responses to these introduced thermal changes across the wearable device and user contact surface(s). More specifically, the observed temperature changes resulting from recharging may be used as thermal inputs based upon which sensors can measure thermal responses and processing circuitry can calculate core temperature (and/or calibrate the system for core temperature calculations). Thus, in one aspect, core temperature calculations may be supported by detecting heating from a power supply during recharging cycles along with corresponding temperature changes throughout the system. Similarly, controlled thermal injections may be provided from inside the wearable device. For example, injecting a controlled thermal excitation may include initiating a recharging power transmission from the power supply to the wearable device, measuring an amount of heat generated by the power supply during this recharging power transmission, measuring other temperatures and/or heat fluxes within the system, and calculating a core temperature therefrom without user intervention.

In general, the battery pack 624 may include components that generate heat during operation. For example, where the battery pack 624 includes a chemical battery or other power storage element, the battery pack 624 may generate heat during charging and discharging due to chemical reactions occurring within the battery as it stores or releases energy, either as a result of the underlying chemical process, as a result of inefficiencies in the battery mechanics, or both. For example, in lithium-ion batteries, the movement of lithium ions between electrodes will generate heat as a byproduct. In addition, internal resistance within a battery will convert some current into heat, and the higher the current flow or resistance, the greater the magnitude of these resistive losses. Where the battery pack 624 receives a charge or provides a charge to a coupled device, the resulting electrochemical and resistive processes will generate heat. As described herein, this heat may be used in a controlled manner along with a suitable heat flux model to estimate core body temperature for a user.

In one embodiment, the system enters the second mode of operation (power supply as controlled heat source) by causing the battery pack 624 to begin charging, a process that results in the generation of heat. In this second mode of operation, the power supply 610 provides a heat source for modifying heat flux through the wearable device 600 in order to estimate a core body temperature of the person wearing the device.

In this second mode of operation, the controller 614 may control the amount of charging power supplied by the battery pack 624 in order to directly or indirectly control the heat generated by the battery pack 624 in its role as a heat source, and the resulting changes in heat can be measured at any suitable location(s), such as the second temperature sensor 612. Thus, in one aspect, the controller 614 places the battery pack 624 into a charging mode that results in the battery pack 624 generating heat. The second temperature sensor 612 can concurrently monitor the temperature of the bottom wall 628 of the power supply 610 where it contacts the housing 602. In one aspect, the second temperature sensor 612 can be used to monitor when this location reaches a selected temperature, e.g., a temperature suitable for evaluating heat flux through the housing 602. Once the bottom wall 628 of the power supply 610 has reached the selected temperature, based on the measurement from the second temperature sensor 612, the controller 614 can determine that a target temperature has been achieved, and may continue to operate the power supply 610 by alternating between charging and not charging in a manner that retains the target temperature for the bottom wall 628 for some predetermined period of time, or for a predetermined duty cycle during which the temperature of the bottom wall 628 is periodically cycled through a series of temperatures. In one aspect, this may include a substantially square wave thermal input cycling between on and off states. However, processing circuitry may also or instead use any suitable control systems (e.g., proportional, integral, or differential control), and/or may vary charging power output with any suitable transfer function in order to maintain a more continuous temperature at or near the target temperature, or time-varying temperature according to a target temperature profile, on the bottom wall 628 of the power supply 610.

The controller 614 can be a standard microcontroller or other discrete and/or programmable processing circuitry of the type commonly employed for implementing programmable instructions in a consumer device or the like. In other embodiments, the controller 614 can be or can implement a state machine or other type of logical system on a programmable device that is capable of carrying out a series of instructions managing the temperature at the second temperature sensor 612 and/or at the bottom wall 628 of the power supply 610 in order to maintain a target temperature for a predetermined amount of time, and/or cycle through a predetermined temperature profile over time.

The bottom wall 628, when heated to a target temperature (or time-varying temperature profile) in this manner, may be used as a heat source that injects heat into the housing 602 and across the circuitry within the housing 602 and toward the body of the wearer. In this way, a heat source can be provided that has a known temperature, in this case the temperature selected by the controller 614 for the bottom wall 628, and that can be held at a selected temperature (or controlled with a time-varying temperature profile) for a selected period of time by the controller 614. As described in more detail herein, this facilitates an estimate of heat flux based on the measurement of a temperature differential between the bottom wall 628 on one hand, and on the other, an opposing surface 632 of the housing 602 that is proximate and thermally coupled to the skin of the wearer and which is at a temperature that can be measured by the first temperature sensor 616, which is thermally connected to the skin of the wearer. The controller 614 may employ the measured temperatures from, for example, the first temperature sensor 616 and the second temperature sensor 612, and optionally an ambient temperature sensor 620, e.g., during calibration or other calculations, to determine the heat flux passing through the wearable device 600.

Suitable thermal sensors for use in the disclosed wearable physiological monitoring systems may include contact and non-contact temperature sensors arranged at one or more locations within the housing 602 or power supply 610. In some embodiments, one or more of the thermal sensors may include thermistors, such as thermistors mounted on a printed circuit board and optionally coupled to a metal contact plate or other thermal contact structure to support stable thermal engagement with the skin. In some embodiments, one or more thermal sensors may include resistance temperature detectors, thermocouples, infrared temperature sensors, clinical-grade temperature sensors, semiconductor-based temperature sensors, or combinations thereof. Where infrared sensing is employed, the thermal sensor may be positioned for optical detection of a target surface, and where contact sensing is employed, the thermal sensor may be positioned directly adjacent to, or in thermal communication with, a corresponding structural component or body surface. In this manner, the disclosed system may employ any suitable arrangement of temperature sensors capable of providing temperature measurements at multiple locations for calibration of the lumped thermal model, determination of heat flux, and estimation of internal body temperature. It will also be appreciated that, while a physical arrangement of temperature sensors is described, the thermal sensors may also or instead include a heat flux transducer 640 or similar component that measures heat flux directly at one or more locations within the system. For example, a variety of suitable commercially available heat flux transducers can be used to generate a voltage proportional to heat passing through the heat flux sensor using a simple formula, e.g., where flux, q, is equal to the output voltage divided by a sensitivity constant for the sensor. While the techniques described herein can usefully leverage existing temperature sensors on circuitry within the housing 602 of a wearable monitor to monitor heat flux, additional components such as the heat flux transducer 640 may also or instead be used to supplement or replace these measurements for use in a core temperature detection system and method as described herein.

In order to accurately calculate core body temperature, thermal properties of the housing 602 may be estimated prior to estimating core body temperature based on heat flux. For example, a calibration process may be performed while the device is not being worn by a user, and during a period when the two surfaces of the housing 602 reach a thermal steady state, such that the first temperature sensor 616 and the second temperature sensor 612 are reading steady temperatures, thereby allowing a determination of the thermal conductivity characterizing the housing 602. The thermal properties of the housing 602 may also or instead be estimated using the first temperature sensor 616 and a measure of the ambient temperature, which may be measured with an ambient temperature sensor 620 or otherwise measured and/or obtained. With these thermal characteristics, the thermal sensors (referring herein to any corresponding combination of the first temperature sensor 616 on the skin side of the housing 602, the second temperature sensor 612 on the bottom wall 628 of the power supply, the third temperature sensor 618 on a side of the housing opposing the first temperature sensor 616, and/or a heat flux transducer 640) can acquire heat flux data for heat flux through the wearable device 600 and the controller 614 can process the heat flux data to determine the core body temperature of a person wearing the wearable device 600. The controller 614 may be a programmable device capable of collecting inputs, such as data from the thermal sensors, and processing these collected inputs to evaluate thermal conductivity (e.g., during calibration) and determine the heat flux passing through the wearable device 600.

The controller 614 may use any suitable technique to determine the core body temperature of the person wearing the wearable device 600. In one aspect, the controller 614 may include processing circuitry that uses a heat flux model based on, e.g., typical thermal conductivity of skin and a known skin temperature (e.g., from the first temperature sensor 616), to derive a measure of core body temperature. However, any suitable technique for determining core body temperature from the measures of heat flux and thermal conductivity derived by the wearable device 600 may be employed, and the specific mathematical technique employed will depend in part on the arrangement and sensitivity of temperature sensors, the degree of control over one or more heat sources, the amount of time available for taking measurements, and the need or desire for accuracy in the final measure of body core temperature. In one embodiment, the core temperature sensor may be realized as a programmable device, such as a microcontroller, a state machine, or other logical processor that is capable of following instructions for processing data such as the heat flux data generated by the thermal sensors to determine the core temperature of the person wearing the wearable device.

In the embodiment depicted in FIG. 6, the controller 614, the thermal sensors, and the processing circuitry 606 for processing biometric data are shown as separate functional elements. However, it will be apparent to those of skill in the art that one or more of these elements may be realized in whole or in part as software processes executing on a microcontroller or other programmable device(s). Further, the depiction in FIG. 6 of these elements as separate functional elements is merely illustrative. In one particular embodiment, the housing 602 includes a microcontroller on a printed circuit board. The printed circuit board may also or instead include sensors such as thermistors, which may be used for measuring temperatures for core body temperature sensing as described herein, as well as other sensors for physiological monitoring of, e.g., cardiac activity, motion, perspiration, and so forth. In embodiments, the power supply 610 may include the controller 614 or another controller. In any case, the logical elements depicted in FIG. 6 can be arranged as needed for a particular application and the requirements thereof, and/or may be adapted for predetermined requirements or design constraints such as cost, power, size, accuracy, and so forth.

While techniques are known in the art for estimating core body temperature based on an externally measured heat flux, the techniques described herein provide significant advantages. In one aspect, the preexisting hardware for a continuously wearable device-a removable and replaceable recharging battery system-provides a controllable thermal excitation source that can be injected into the recharging circuitry of the device for thermal modeling and inferencing. Thus, instead of passively inferring core temperature only from skin temperature and ambient temperature, the system can drive heat into the thermal stack and observe the resulting temperature response. This supports the detection of thermal parameters and calibration of a thermal model for use in calculating core body temperature. As a further advantage, the removable battery pack provides additional temperature measurement and/or heating locations that are physically distinct from the skin-facing sensor(s) in the wearable monitor. This provides additional information about the temperature gradient across the stack, which supports the measurement of heat flux and resolution of a lumped thermal model with more nodes and/or better constraints.

Furthermore, the removability of the battery pack provides additional configurations for improved calibration and characterization of lumped characteristics. For example, measurements can be taken in numerous configurations, such as with the wearable device off-body, the battery pack separately, the wearable device off-body with the battery pack attached, the wearable device on-body without the battery pack, the wearable device on-body with the battery pack without active heat injection, and the wearable device on-body with the battery pack and active heat injection, and/or the wearable device off-body with the battery pack and active heat injection. This variety of potential configurations permits isolation and identification of thermal resistance or other parameters attributable to each component separately, which means that a larger system of equations can be generated and resolved to distinguish the thermal behavior of each individual component (battery pack and wearable device) as well as the group of components together, rather than modeling the battery and device as a single undifferentiated block. Thus, the removable battery pack is not simply a heat source for calibration of a model of the device. The removable battery pack can be independently modeled and characterized as a thermal system to permit improved isolation and accuracy of measurement of the parameters of the device.

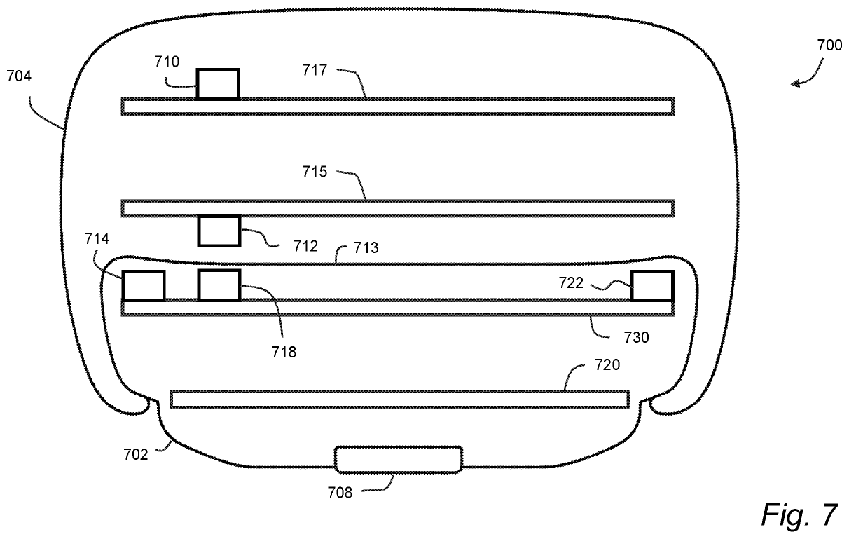

FIG. 7 depicts a cross-section of a physiological monitoring device with core temperature sensing. In general, the device 700 may be any of the wearable devices described herein, and may include a housing 702 and a power supply 704. The housing 702 can be a housing supporting any of the wearable physiological monitors described herein. As described herein, the housing 702 may include or be coupled to a strap (not shown), ring, compression garment pocket, or other hardware for securing the housing 702 to a body for use in monitoring. Sensors in the housing 702 may include a first temperature sensor 708 for measuring a skin temperature of a wearer of the device 700. For example, the first temperature sensor 708 may be positioned on or in the housing 702 to make thermal contact with the skin of a user wearing the device. Thermal contact, as that term is employed herein, will be understood to mean contact, whether direct or indirect, between two bodies that allow for a sensor on one body to measure the temperature of the other body. For example, in one embodiment, the first temperature sensor 708 may include a thermistor disposed on a printed circuit board and having a metal contact plate or other thermal contact structure so that the thermistor can be in direct and/or stable thermal contact with the skin (e.g., with the metal plate positioned on the housing where the metal plate will directly contact the skin of the person wearing the device 700) in a manner that permits measurement of skin temperature. In general, the first temperature sensor 708 may include a thermistor or any of the other temperature-sensing devices described herein.

The housing 702 may include a first circuit board 720 above the first temperature sensor 708 containing electronics for biometric sensing and/or core temperature monitoring as described herein. In general, heat generated by any of the circuitry within the housing 702 may also be accounted for as a heat source when modeling for heat flux within and around the device 700. Arranged above the first circuit board 720 (relative to the skin or other contact surface) may be a second circuit board 730, which may include a printed circuit board having a microcontroller programmed to control and execute the calibration and core temperature measurement processes described herein, e.g., using the power supply 704 and thermal sensors. The second circuit board 730 may include a second temperature sensor 718. An ambient temperature sensor 722 may be positioned within the housing 702 for direct or indirect contact with the ambient environment surrounding the device 700 to permit measurement of the ambient temperature. In general, the ambient temperature sensor 722 may monitor the temperature of the ambient environment about the device 700, or a surface of the device 700 exposed to the ambient environment. The housing 702 may also contain a humidity sensor 714 capable of measuring humidity in the ambient environment. This may be used, for example, to more accurately model the heat transfer properties of the ambient environment, or otherwise improve estimates of heat flux and/or core temperature as described herein.

The power supply 704 may be a removable power supply that can slide onto the housing 702 and removably and replaceably connect with the housing 702. The power supply 704 may connect mechanically to the housing 702 in a manner that allows for a thermal coupling and heat flow between the power supply 704 and the housing 702. The power supply 704 may also include one or more electrical contacts (not shown) for electronically coupling to the housing 702 and components therein, and/or the power supply 704 may include circuitry for wireless data and power transfers between the power supply 704 and the housing 702.

The thermal connection between the power supply 704 and the housing 702 allows for thermal energy generated by the power supply 704 to transfer to the housing 702. The power supply 704 may include a third temperature sensor 712 positioned proximal to or in contact with a lower surface 713 of the power supply 704. When the power supply 704 is placed for use on the housing 702, the lower surface 713 of the power supply 704 may in turn be thermally coupled to the housing 702 for thermal flow therebetween. As depicted, the third temperature sensor 712 may be on a lower side of a third circuit board 715 within the power supply 704. The power supply 704 may also include a fourth circuit board 717 positioned above the third circuit board 715 to support additional electronics, sensors, and so forth. The fourth circuit board 717 may include a fourth temperature sensor 710. Arranged in this manner, the collection of thermal sensors can instrument temperature at or near opposing surfaces of the housing 702 and power supply 704, more specifically for estimation of heat flux therethrough. It will be appreciated that FIG. 7 is for purposes of illustration, and the various processors, sensors, and other electronics and components of the power supply 704 and housing 702 may more generally be positioned in a variety of locations consistent with measuring core temperature as described herein.

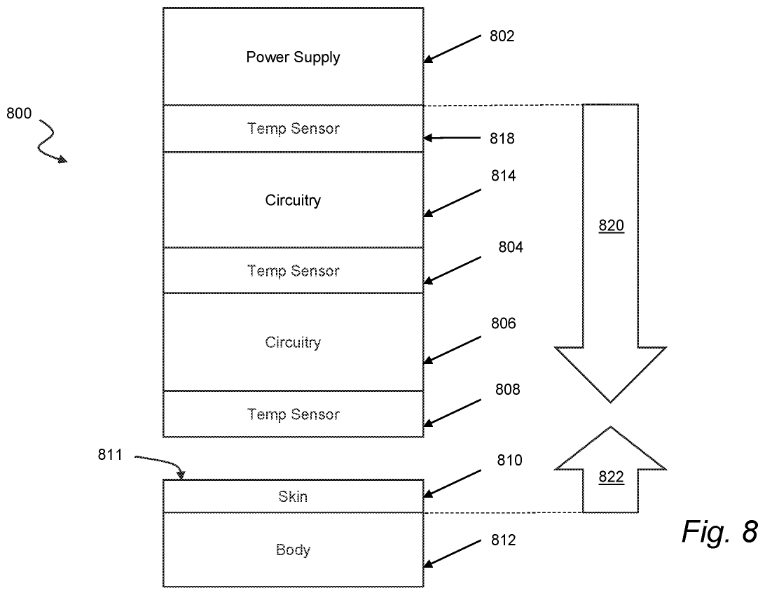

FIG. 8 depicts a flow of heat through a system including a wearable device and a removable power supply. The system 800 may include any of the wearable devices and related hardware described herein. The system 800 may generally form a thermal stack that includes a power supply 802, a first temperature sensor 818, first circuitry 814, a second temperature sensor 804, second circuitry 806, and a third temperature sensor 808. This stack of thermal elements is adjacent to the skin 810 (and the body 812, which underlies the skin 810), and the system 800 may generally be positioned on the skin 810 at a location where core body temperature, e.g., the stable interior temperature within the body 812, is to be measured. As depicted, the power supply 802 provides a first heat source, with heat flowing toward the third temperature sensor 808 as depicted by a first arrow 820 when there is a corresponding heat gradient resulting from heat generated by the power supply 802, and the body 812 provides a second heat source, with heat flowing toward the third temperature sensor 808 as depicted by a second arrow 822.

As described herein, the power supply 802 can act as a controllable heat source that can be maintained at a selected (or time-varying) temperature for a predetermined period of time, with a temperature that can be measured at its source by the first temperature sensor 818. The first temperature sensor 818 may, for example, be coupled to or positioned adjacent to the power supply 802, e.g., positioned on a surface where the power supply 802 contacts other elements of the system 800 in the thermal stack. The third temperature sensor 808 may be positioned adjacent to the skin 810 to measure a temperature at the skin surface 811 when the system 800 is placed for use in contact with the skin 810. The second temperature sensor 804 may be positioned at any suitable location within the system 800 between the two opposing heat sources of the body 812 and the power supply 802. Applying general thermodynamic principles, when the temperature on two sides of a region, such as the thermal stack of the system at the first temperature sensor 818 and the third temperature sensor 808, is known and in a steady state, the thermal conductivity of the material can be measured based on the temperature difference between the two surfaces. It will be understood that the system may contain additional heat sources. For example, circuitry within the housing, and/or heat losses during charging of the housing by the power supply 802, may create additional heat within the system 800. In general, these separate heat sources may be identified and modeled as elements of a lumped thermal system using the various calibration strategies described herein, and may be applied to parameterize the overall system model for calculation of the core temperature based on measurements of temperature and/or heat flux from the thermal sensing components of the system 800.

In general, a single heat flux model may be devised that assumes the body core and skin surface are separated by a layer of tissue with a specific thermal resistance. In this case, the simplified formula for core temperature based on Fourier's Law is:

T_{c}=Tₛₖᵢₙ+(q·Rₛₖᵢₙ)
where:
T_{c}=Core Body Temperature
Tₛₖᵢₙ=Skin Temperature measured by the skin sensor
q=Measured heat flux (using heat flux transducer or second temperature sensor)
Rₛₖᵢₙ=Thermal resistance of skin (measured in calibration)

In order to address various challenges such as accurately measuring surface heat flux and accounting for variations in skin resistance, multiple temperature sensors can be used in a stack that is separated by thermal resistors. In this case, a dual heat flux model may be used to calculate heat flux based on the difference in temperatures across a known insulator:

$${T}_{c} = {T}_{1} + ( \frac{{R}_{skin}}{{R}_{insulator}} ) ( {T}_{1} - {T}_{2} )$$

Where T₁ is the skin temperature, T₂ is the opposing temperature (e.g., the opposite side of a device contacting the skin), and the thermal resistance metrics for skin and the insulator between the two temperature measurement locations can be determined using calibration steps as described herein. In other embodiments, the skin thermal resistance can also be estimated based on typical ranges, although it is possible to obtain a more precise estimate using the multi-step calibration techniques described herein. As further described herein, this general model may be improved by including a thermal model (e.g., lumped thermal resistance) for the battery pack and the controlled thermal excitation temperature provided by the heated battery pack during charging.

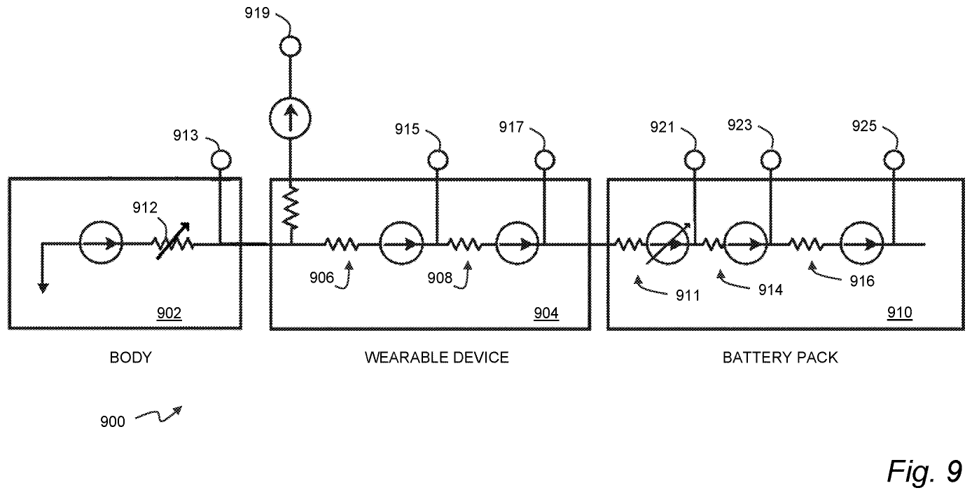

In some embodiments, the disclosed system models heat transfer between a body interior region and a controllable heat source of a wearable device using a full lumped thermal model of a thermal stack extending through the skin, the wearable monitor, and a removable battery pack. The full model can represent the body as a first element providing a body-side heat flux through a first thermal resistance of the skin to a skin-surface node, and can further represent the wearable device as a compound element including a monitor portion and a power-supply portion, each of which can include one or more additional lumped thermal resistances and intermediate temperature nodes corresponding to temperature measurement locations within the thermal stack, for example, as illustrated in FIG. 9. In this manner, the model can use a plurality of measured temperatures, including a skin-facing temperature, one or more temperatures within the wearable monitor, and one or more temperatures within the battery pack, together with corresponding heat-flux relationships across physical bodies modeled as serially arranged thermal resistances, to characterize heat flow from the body and heat injected from the battery pack when the battery pack is operated as a controllable heat source. In some implementations, once individual thermal parameters of the full model have been characterized through calibration measurements, the full model can be reduced by assuming, for a selected measurement interval, that heat flow through the monitor portion and the power-supply portion is substantially one-dimensional, that parasitic heat transfer through straps, supports, and other external structures is negligible, that internal heat losses apart from the controlled heat source are negligible or separately accounted for, and that selected portions of the wearable monitor and the battery pack can be combined into equivalent thermal resistances. Under these assumptions, multiple serial resistances and intermediate temperature nodes of the full model can be collapsed into a smaller set of equivalent resistances and temperatures, thereby yielding a reduced model that preserves the controlled thermal excitation at the battery-pack side, the measured skin-side temperature, and the resulting heat flux through the thermal stack for estimation of internal body temperature.

For example, in some embodiments, the wearable physiological monitoring system estimates internal body temperature using a lumped thermal model of the thermal stack extending from a body interior region, through the skin, through the wearable monitor, and through the removable battery pack, where the controller receives temperature measurements from thermal sensors during a controlled thermal excitation produced by operating the battery pack as a controllable heat source. In one implementation, the reduced lumped model may be expressed as:

$${T}_{core} = {T}_{skin} + {R}_{skin} * ( \frac{( {T}_{exc} - {T}_{skin} )}{( {R}_{BP} + {R}_{dev} )} )$$

where:
T_{core} is the estimated core temperature,
T_{exc} is the controlled thermal excitation temperature associated with the heated battery pack or other controllable heat source,
Tₛₖᵢₙ is the measured skin-side temperature,
R_{BP} is the lumped thermal resistance of the battery pack portion of the stack,
R_{dev} is the lumped thermal resistance of the wearable monitor portion of the stack,
Rₛₖᵢₙ is the lumped thermal resistance between the skin surface and the body core, and
   Similarly, the heat flux, q, through this thermal circuit (including the battery pack and wearable monitor) may be expressed as:

$$q = \frac{( {T}_{exc} - {T}_{skin} )}{( {R}_{BP} + {R}_{dev} )}$$

This may be useful, for example, where the system includes a heat flux transducer for direct measurement of flux across some portion of the physical system. Of course, this general model may be expanded to any number of thermal nodes, and/or simplified to one or more nodes based on the data that is available for test-time calibration and run-time core temperature measurement. The processing circuitry may determine one or more of the temperatures and thermal resistances from calibration measurements, ambient measurements, exponential-rise fitting, or combinations thereof, and then solve the lumped thermal model to estimate the internal body temperature from temperature data collected at multiple locations of the wearable monitor.

In some embodiments, the disclosed wearable temperature estimation system is calibrated by first calibrating one or more temperature sensors at ambient temperature and storing corresponding calibration values in memory, thereby providing at least a one-point calibration for the temperature sensors. After sensor calibration, the wearable strap may be placed on the human body and temperatures may be measured with dissipation maintained close to zero in order to establish a body baseline and characterize thermal properties of the strap portion of the system. A battery pack may then be coupled to the strap, and initial temperature measurements may be obtained with charging inactive, where an exponential rise model is used to estimate heat flux and to characterize a thermal resistance associated with the battery pack under low-heat-flow conditions. Wireless charging may then be initiated to provide a controlled thermal excitation, and additional temperature measurements may be obtained while the exponential rise model is used to estimate skin temperature and solve for remaining variables of a lumped thermal model of the thermal stack. More generally, any techniques for estimating the thermal resistance of various nodes of a lumped thermal model may be used to calibrate a system for use in measuring core temperature as described herein. Using the resulting calibrated sensor values, baseline measurements, estimated heat flux, and thermal resistance values, the processing circuitry may calculate an internal body temperature using the techniques described herein. In some development or calibration procedures, the battery pack may also be arranged in different orientations, including a reversed orientation, to further characterize the thermal behavior of the system.

FIG. 9 depicts a heat flow model for a wearable device, such as any of the wearable devices described herein, positioned for monitoring on a user. In general, a model 900 for heat flow may model the human body as a first thermal element 902 that has a first heat flux representing heat from the body core that passes through a first thermal resistance 912 of the skin. The heat from within the body passes through the thermal resistance of the skin, generating a skin temperature 913 that can be measured at the epidermis using a temperature sensor such as any of those described herein.

The wearable device is shown as a second thermal element 904, which may include two separate sub-elements: a first element 906 corresponding to a first circuit board in the wearable device (e.g., such as any described herein) and a second element 908 corresponding to a second circuit board in the wearable device. Although not mathematically necessary, this approach to modeling conveniently maps the nodes of the thermal network to the physical arrangement of the wearable device, as well as the probe locations where temperature data is available. For example, the skin temperature 913 may be measured using a contact thermistor or the like of the wearable device, while a first internal temperature 915 of the wearable device may be obtained from a temperature sensor on a circuit board of the wearable device. Similarly, a second internal temperature 917 of the wearable device, e.g., at a surface of the wearable device contacting the battery pack, may be obtained from another temperature sensor on a second circuit board of the wearable device or otherwise positioned for sensing at a suitable location next to the battery pack, or otherwise positioned between the thermistor for the first internal temperature 915 and the battery pack. Similarly, an ambient temperature 919 may be measured, e.g., using an ambient temperature sensor of the wearable device, and used to calibrate the lumped thermal model, and/or to perform core temperature calculations.

The battery pack may also be modeled as a third thermal element 910. In an embodiment, the battery pack includes sub-elements, such as a third element 911 for the battery circuit board, which includes its own thermal resistance and a third internal temperature 921 that can be measured, e.g., with a temperature sensor on the circuit board, and a fourth element 914 for the power supply, which also includes its own thermal resistance and a fourth internal temperature 923 that can be measured, e.g., with a temperature sensor on or near the power supply itself. Finally, the model for the battery pack may include a fifth element 916 to account for heat loss from the battery pack to the environment, which may be measured, e.g., with a surface temperature sensor 925 or the like.

In general, these elements may be characterized with a system of, e.g., five equations in seven variables (assuming no heat loss). These equations can be fully solved, e.g., by measuring a number of steady-state conditions, e.g., with the monitor on and off the body, with and without the battery pack, and with or without heat excitation from the battery pack. It will be appreciated that simpler or more complex models may be created, although the above model maps well to a system that includes a device with two circuit boards, each having its own temperature sensor, along with a battery pack that can be controlled to provide heat excitation. While steady-state modeling simplifies calculations, it will be appreciated that a time constant can also be resolved for various elements of the model based on, e.g., the rise time of a temperature at a location in response to a known heat input from the battery pack or the like, which may be derived from a time series of temperature measurements at that location following the thermal input. As a significant advantage, resolving time constants in this manner can facilitate speedier calculation of core temperature after calibration. Rather than waiting for a steady-state condition across the system, the steady state can be inferred based on short-term impulse responses to step changes in heat input, from which long-term results can be more quickly estimated.

According to the foregoing, a power supply, such as the battery pack or other heating element, may be used to controllably inject heat into the system. With a number of additional assumptions, such as no heat flux from the additional hardware controlling or physically retaining the wearable device, and no other sources of heat loss within the system, the depicted model can be reduced to a number of equations and variables that can be unambiguously solved for heat flux from the body, which represents the core temperature. In one aspect, the heat source may be controlled to equalize the temperature across sensors, at which point the heat source temperature should equal the core temperature. In another aspect, heat may be applied in a step function or impulse function, and the change through the stack of temperature sensors can be monitored to infer the core temperature on the other end of the thermal stack from the heat source based on known or estimated rise times. More generally, a variety of calibration and drive techniques may be adapted for measuring core body temperature based on an external, applied heat source using a recharging battery for a wearable device.

In another aspect, the heat from the power supply may also cause heating within the wearable device, e.g., as an internal charging circuit converts wireless power from the battery pack into power to charge an internal battery. This separate (but dependent) heat source may also be modeled where the response is known (or resolvable through calibration) and where the additional heat affects steady-state temperature within the system in a manner that will impact core temperature calculation.

In one aspect, the system can be calibrated by measuring temperatures across the system without active heat injection, e.g., to provide temperatures across the model under ambient conditions, thermal resistances, and so forth. In order to provide additional measurements to sufficiently constrain a calculation of core temperature, other measurements may also be taken, such as conditions while the wearable monitor is off-body, on-body without battery pack, on-body with battery pack and without active heat injection, on-body with battery pack and heat injection, and so forth. Similarly, the battery pack (the third thermal element 910 of the model 900) and the wearable monitor (the second thermal element 904 of the model 900) may be physically separated, and additional measurements may be taken, e.g., to characterize each element of the model independently.

Once sufficient calibration measurements have been taken to resolve the parameters of the lumped thermal model, e.g., thermal conductance or resistance for each node in the model, the wearable device and battery pack may be positioned on the body for a core temperature measurement. In one aspect, the device and battery may be positioned on the body without heat injection, and an exponential rise model or the like may be used to estimate flux parameters. Heat injection may then be initiated, e.g., by initiating wireless charging through the battery pack, and an exponential rise model or the like may be used to solve for remaining variables and calculate a core temperature. Once the device is positioned and sufficient calibration measurements have been obtained to characterize parameters of the thermal flow model, additional core body temperature measurements may be taken as needed or desired by positioning the battery pack on the monitor and cycling through controlled wireless charging to inject heat and measure responses thereto.

It will be understood that a variety of different circuit models or other models or techniques may be used to characterize the thermal behavior and properties of the system. Other properties and features of a core temperature measurement system as contemplated herein will also be appreciated from the foregoing. For example, as described above, the battery pack may be used as a controllable heat source when in a charging mode. However, other systems and components may also or instead be used to generate heat, such as light-emitting diodes, haptics, and electronic circuitry. Thus, for example, by aggressively discharging a battery on the battery pack or the device, additional controlled heat may be generated. In another aspect, measured parameters may include one or more of wearable device temperature (at one or more locations), wearable device temperature rate of change, battery pack temperature (at one or more locations), battery temperature rate of change, battery pack power dissipation, wearable device power dissipation, and so forth. Other parameters may be usefully modeled based on known physical properties or based on calibration procedures such as those described herein. For example, this may include air temperature, skin surface temperature, blood perfusion (related to temperature change), body thermal impedance, body heat stress, device component temperatures, and core temperature. In another aspect, the battery pack may be used as a calibration tool that includes a memory storing tracked or measured parameters for use with the same wearable and user in order to support subsequent core temperature measurements.

It will also be appreciated that, while the lumped model is derived from physical insights that provide a useful shortcut to modeling, similar results may also be achieved, e.g., with a large data set of heat impulses and temperature measurements labeled with clinical measurements of core body temperature. With a data set formed in this manner, a variety of machine learning strategies may be employed to infer core body temperature from new heating profiles and temperature measurements. Thus, in some embodiments, instead of, or in addition to, estimating internal body temperature with a lumped thermal model, the wearable physiological monitoring system may estimate internal body temperature with a machine learning model trained on a data set including reference internal body temperature measurements associated with known thermal excitation conditions and corresponding temperature responses measured at multiple locations of the wearable monitor and the battery pack. The machine learning model may be trained using supervised learning based on training records in which a controlled heat input, a resulting temporal temperature response of the thermal stack, ambient conditions, and a corresponding reference internal body temperature are known. In some embodiments, the training data may include temperature measurements obtained from one or more skin-facing thermal sensors, one or more thermal sensors disposed within the wearable monitor, and one or more thermal sensors disposed within the battery pack, and may optionally include feature data that characterizes device charging state, charging power, battery temperature, heat injection timing, user activity, ambient temperature, humidity, and so forth, or combinations thereof. The machine learning model may include a regression model, a neural network, a recurrent neural network, a temporal convolution model, a transformer model, an ensemble model, or another statistical learning model configured to map the controlled thermal excitation and the measured temperature response to an estimated internal body temperature. In some embodiments, the machine learning model may be trained to infer internal body temperature directly from the measured temperature response, or may be trained to estimate one or more intermediate variables, such as heat flux, thermal resistance, or calibration parameters, that are then used alone or in combination with the lumped thermal model. In this manner, the machine learning model may learn relationships between known heat injections and corresponding multi-location temperature responses from a large population-level and/or subject-specific data set, thereby enabling estimation of internal body temperature without requiring explicit solution of the lumped thermal model.

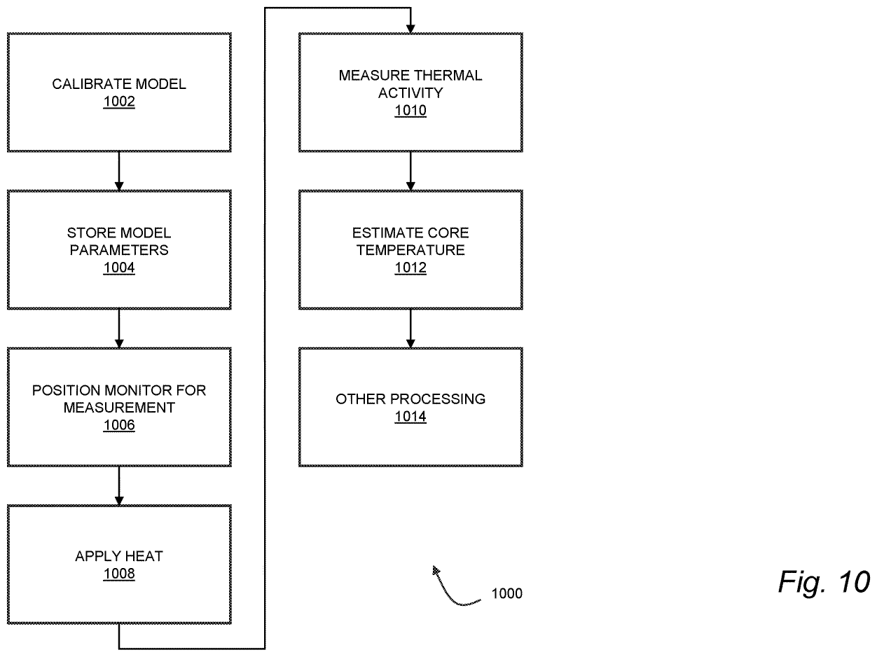

FIG. 10 shows a method for estimating core temperature with a wearable device. In some embodiments, the method 1000 may be implemented by computer-executable code embodied in a non-transitory computer-readable medium and executed by one or more computing devices, such as a controller of the wearable monitor, a controller of the recharging battery, or other processing circuitry or combinations thereof.

As shown in step 1002, the method 1000 may include calibrating a lumped thermal model for a wearable monitor, such as any of the monitors described herein, based on one or more temperature measurements obtained under controlled conditions including predetermined configurations of the wearable monitor and the recharging battery. In some embodiments, obtaining measurements under the controlled conditions may include obtaining one or more temperature measurements while the recharging battery is coupled to the wearable monitor and the wearable monitor is not worn by the user. In some embodiments, the controlled conditions may additionally or alternatively include obtaining one or more temperature measurements while the wearable monitor is placed on the skin for use without the recharging battery, placed for use on the skin with the recharging battery, and/or placed for use on the skin with the recharging battery while the recharging battery is providing power.

In one aspect, the calibration procedure may be orchestrated using an additional user device. For example, a smartphone or other device may provide a user interface that guides a user through one or more calibration steps during which a user is instructed to put the device in a predetermined state (e.g., on wrist, with battery pack attached), and then the device is cycled through a desired test protocol, e.g., by injecting heat with the battery pack and then measuring temperature changes at one or more temperature sensor locations over time. The user may then be instructed to place the device in a next state (e.g., off wrist, with battery pack attached) and so forth until sufficient calibration data has been obtained to resolve a set of equations characterizing a lumped thermal model for the wearable monitor (and/or skin and tissue, battery pack, and so forth, where these are used for live core temperature calculations). In one aspect, this may be performed with a paired device and battery pack before shipping or otherwise releasing to a customer. In another aspect, this may be performed by a user prior to core temperature measurements. This latter approach advantageously permits accurate characterization of a user's skin thermal conductance, which can be highly variable from person to person, and may also vary over time for a particular individual in response to hydration, sweat, tissue composition, blood flow, and so forth. More generally, any technique for calibrating a lumped thermal model or the like for use in estimating a core temperature may be used to obtain model parameters or other calibration values as described herein.

Other calibration techniques may also or instead be used. For example, in one aspect, physiological states such as sleep states may be used to support calibration. Sleep states have known correlations to core temperature. Where these changes provide predictable, recurring thermal changes on one end of the lumped thermal model, e.g., a −0.5 °C core temperature drop in core temperature at sleep onset, this provides a thermal step function on the user side that provides a reference point for calibration/validation measurements without external power supplies or additional temperature sensors.

As shown in step 1004, the method 1000 may include storing parameters for a lumped thermal model of the wearable monitor. In some embodiments, the stored parameters may include a thermal conductivity of at least one of a body of the user, the wearable monitor, and the recharging battery, and/or individual sub-components thereof, such as thermal conductivity of individual circuit boards or other assemblies, and thermal conductivity from one or more of the components or sub-components to an ambient environment. Other properties may, for example, include thermal resistances, thermal circuit time constants, or other model parameters associated with heat transfer through the thermal stack. The current model may also or instead be stored for use in calculating core temperatures, as well as any calibration models or individual component models useful in calibrating or otherwise using a core temperature estimation system as described herein.

As shown in step 1006, the method 1000 may include positioning a first side of the wearable monitor for temperature measurements. For example, this may include positioning the wearable monitor against the skin of the user with the recharging battery or other power supply removably coupled thereto, e.g., on a second side of the wearable monitor opposing the first side. The wearable monitor may be coupled to the recharging battery for power and communications using wired connections, a wireless interface, or some combination of these.

As shown in step 1008, the method 1000 may include applying heat to the wearable monitor, e.g., by controlling the recharging battery, while removably coupled to the wearable monitor, to provide a controlled thermal excitation to a surface of the wearable monitor. In some embodiments, in which the wearable monitor is wirelessly coupled to the recharging battery, initiating the controlled thermal excitation may include transmitting a control signal from the controller of the wearable monitor to the recharging battery through a wireless communications interface between the wearable monitor and the recharging battery.

As shown in step 1010, the method 1000 may include measuring thermal activity while providing the controlled thermal excitation to the wearable monitor. For example, this may include measuring any temperatures and/or heat fluxes within a system including the wearable monitor. For example, the method 1000 may include measuring a skin temperature of the user where the wearable monitor contacts the skin of the user. The method 1000 may also or instead include measuring a heat flux through the wearable monitor between the first side contacting the skin and the second side contacting the recharging battery, or otherwise positioned between the skin and the recharging battery in a location that permits an evaluation of heat flux therebetween. In another aspect, measuring thermal activity may include measuring temperature changes over time, which facilitates the measurement and analysis of rise times or other properties of a dynamic thermal system.

As shown in step 1012, the method 1000 may include estimating the core body temperature of the user by applying the skin temperature and the heat flux to the lumped thermal model. This may include applying the measured thermal activity to any of the corresponding models described herein in a manner that permits an estimation of core temperature based on point temperature measurements and/or heat flux measurements.

In general, estimating the core body temperature may include adjusting an estimate of the core body temperature using any available sensed or modeled data or information. For example, in one aspect, the method 1000 may include continuously measuring ambient temperature and continuously adjusting a core temperature calculation based on modeled heat losses into the ambient environment. In another aspect, the core body temperature may be adjusted based on user activity. For example, a wearable monitor may measure motion, heart rate, or the like and determine a level of physical activity or exertion for a user. This measurement may be used to adjust the core body temperature calculation to account for surface temperature increases attributable to exertion, e.g., by scaling the measured skin temperature down when activity levels are high, or otherwise adjusting model calculations to account for temperature increases resulting from user activity. In another aspect, core temperature calculations may be paused or avoided during highly dynamic intervals, e.g., where a user is exercising vigorously, where initial model assumptions are potentially invalid, or where temperature is otherwise known to deviate significantly from sedentary core temperature measurements using the techniques described herein.

In another aspect, ambient conditions may be sensed and used to adjust core temperature calculations. For example, ambient humidity may be used to improve modeling of environmental heat dissipation. In another aspect, a sweat monitor may be used to detect and model properties of the skin interface, e.g., to adjust modeling to account for changes in skin thermal conductivity, evaporative cooling, and so forth.

As shown in step 1014, the method 1000 may include other processing. For example, the method 1000 may include storing a calculated core body temperature, e.g., on the wearable device, on the recharging power supply, on another user device (such as a laptop or smartphone), on a remote server, and so forth. Other processing may also or instead include displaying the core body temperature to a user, e.g., on a display of the wearable device, the recharging power supply, or another user device. In another aspect, additional processing may include identifying potential interventions and transmitting related information to a user. This may, for example, include an active intervention during vigorous exercise where core body temperature appears to be rising, or a recommendation for more rest when an increase in core body temperature during a sedentary interval, e.g., while sleeping or over an extended immobile period, indicates an onset of illness. More generally, any inferences about health and/or potential interventions, coaching, or remediations that are suggested based on variations of the measured core body temperature from a predicted or normal core body temperature may be used to initiate corresponding notifications, recommendations, or other coaching and interventions to the user.

The above systems, devices, methods, processes, and the like may be realized in hardware, software, or any combination of these suitable for a particular application. The hardware may include a general-purpose computer and/or dedicated computing device. This includes realization in one or more microprocessors, microcontrollers, embedded microcontrollers, programmable digital signal processors or other programmable devices or processing circuitry, along with internal and/or external memory. This may also, or instead, include one or more application-specific integrated circuits, programmable gate arrays, programmable array logic components, or any other device or devices that may be configured to process electronic signals. It will further be appreciated that a realization of the processes or devices described above may include computer-executable code created using a structured programming language such as C, an object-oriented programming language such as C++, or any other high-level or low-level programming language (including assembly languages, hardware description languages, and database programming languages and technologies) that may be stored, compiled, or interpreted to run on one of the above devices, as well as heterogeneous combinations of processors, processor architectures, or combinations of different hardware and software. In another aspect, the methods may be embodied in systems that perform the steps thereof and may be distributed across devices in a number of ways. At the same time, processing may be distributed across devices such as the various systems described above, or all of the functionality may be integrated into a dedicated, standalone device or other hardware. In another aspect, means for performing the steps associated with the processes described above may include any of the hardware and/or software described above. All such permutations and combinations are intended to fall within the scope of the present disclosure.

Embodiments disclosed herein may include computer program products comprising computer-executable code or computer-usable code that, when executing on one or more computing devices, performs any and/or all of the steps thereof. The code may be stored in a non-transitory fashion in a computer memory, which may be a memory from which the program executes (such as random-access memory associated with a processor), or a storage device such as a disk drive, flash memory, or any other optical, electromagnetic, magnetic, infrared, or other device or combination of devices. In another aspect, any of the systems and methods described above may be embodied in any suitable transmission or propagation medium carrying computer-executable code and/or any inputs or outputs from same.

It will be appreciated that the devices, systems, and methods described above are set forth by way of example and not of limitation. Absent an explicit indication to the contrary, the disclosed steps may be modified, supplemented, omitted, and/or re-ordered without departing from the scope of this disclosure. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party, such as a remote user, a remote processing resource (e.g., a server or cloud computer), or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

More generally, while particular embodiments have been shown and described, it will be apparent to those skilled in the art that various changes and modifications in form and details may be made therein without departing from the spirit and scope of this disclosure and are intended to form a part of the invention as defined by the following claims.

What is claimed is:

1. A system comprising:
a wearable monitor including a housing configured to be worn against a skin of a user, the housing including a first temperature sensor positioned to obtain a temperature at the skin;
a power supply removably and replaceably coupled to the housing of the wearable monitor, the power supply including a second temperature sensor, the power supply operable in a first mode as a recharging battery to provide electrical power to recharge the wearable monitor, and the power supply operable in a second mode to generate controlled heat for injection through a thermal stack extending from the power supply to the skin of the user; and
processing circuitry configured to:
operate the power supply in the second mode to provide a controlled thermal excitation,
receive temperature information from the first temperature sensor and the second temperature sensor indicative of a temperature response of the thermal stack to the controlled thermal excitation,
determine a heat flux through the thermal stack based on the temperature response, and
estimate an internal body temperature of the user based on the heat flux through the thermal stack.

2. The system of claim 1, wherein the processing circuitry is configured to estimate the internal body temperature using a lumped thermal model calibrated based on a number of measurements of thermal flux through the housing under a number of operating conditions.

3. The system of claim 2, wherein the lumped thermal model represents the user, the housing, and the power supply as elements of the thermal stack having corresponding thermal resistances.

4. The system of claim 2, further comprising a temperature sensor positioned to measure an ambient temperature, wherein the lumped thermal model is calibrated based at least in part on a second heat flux from the housing to an ambient environment.

5. The system of claim 1, wherein the processing circuitry is configured to estimate the internal body temperature based on a time constant derived from the temperature response.

6. The system of claim 5, wherein the processing circuitry initiates one or more heat injections from the power supply and calculates the time constant based on a rise time for a temperature at one or more of the first temperature sensor and the second temperature sensor.

7. The system of claim 1, wherein the processing circuitry is configured to detect a steady state of the temperature response to the controlled thermal excitation and to estimate the internal body temperature based on one or more temperature measurements at the steady state.

8. The system of claim 1, wherein the power supply includes a second sensor for measuring a temperature and the second sensor is disposed on a second side of the processing circuitry opposite a first side of the processing circuitry facing the skin of the user.

9. The system of claim 1, wherein the processing circuitry stores one or more parameters representative of nodes of a lumped thermal model for estimating the internal body temperature, the lumped thermal model including at least a first thermal conductivity associated with the power supply and a second thermal conductivity associated with the housing.

10. The system of claim 1, further comprising a third temperature sensor on a second side of the housing opposing the first temperature sensor and a fourth temperature sensor on a second side of the power supply opposing the second temperature sensor.

11. The system of claim 1, wherein the housing includes a heat flux transducer for measuring a housing heat flux therethrough.

12. A computer program product for estimating an internal body temperature of a user with a wearable monitor, the computer program product comprising computer-executable code embodied in a non-transitory computer-readable medium that, when executing on one or more computing devices, causes the one or more computing devices to perform the steps of:
storing parameters for a lumped thermal model of the wearable monitor;
controlling a recharging battery removably coupled to the wearable monitor to provide a controlled thermal excitation to the wearable monitor;
while providing the controlled thermal excitation to the wearable monitor, measuring a skin temperature of the user where the wearable monitor contacts a skin of the user;
while providing the controlled thermal excitation to the wearable monitor, measuring a heat flux through the wearable monitor between a surface of the wearable monitor contacting the skin and a location between the surface of the wearable monitor and the recharging battery; and
estimating the internal body temperature of the user by applying the skin temperature and the heat flux to the lumped thermal model.

13. The computer program product of claim 12, wherein the parameters for the lumped thermal model include a thermal conductivity of at least one of a body of the user, the wearable monitor, and the recharging battery.

14. The computer program product of claim 12, further comprising code that causes the one or more computing devices to perform the step of calibrating the lumped thermal model based on one or more temperature measurements under controlled conditions including predetermined configurations of the wearable monitor and the recharging battery.

15. The computer program product of claim 14, wherein the one or more temperature measurements include one or more temperature measurements while the recharging battery is coupled to the wearable monitor and the wearable monitor is not worn by the user.

16. The computer program product of claim 14, wherein the one or more temperature measurements include one or more temperature measurements while the wearable monitor is placed for use on the skin without the recharging battery.

17. The computer program product of claim 12, wherein the wearable monitor is wirelessly coupled to the recharging battery for power and communications.

18. The computer program product of claim 17, further comprising code that causes the one or more computing devices to perform the step of initiating the controlled thermal excitation from the wearable monitor through a wireless communications interface between the wearable monitor and the recharging battery.

19. A method for estimating an internal body temperature of a user, the method comprising:
positioning a first side of a wearable monitor against a skin of the user;
removably coupling a power supply to a second side of the wearable monitor opposing the first side;
while providing a controlled thermal excitation to the wearable monitor with the power supply coupled to the wearable monitor, performing the steps of:
measuring a skin temperature of the user where the wearable monitor contacts the skin, and
measuring a heat flux through the wearable monitor; and
estimating the internal body temperature of the user based on the skin temperature and the heat flux.

20. The method of claim 19, wherein measuring the heat flux includes measuring the heat flux with a plurality of temperature sensors within the wearable monitor.
